(19) United States
(12) Patent Application Publication
(10) Pub. No.: US 2023/0116707 A1
(43) Pub. Date: Apr. 13, 2023

(54) DETECTION OF NOVEL DEGRADATION-RELATED INTERACTIONS

(71) Applicants: Orionis Biosciences, Inc., Waltham, MA (US); Orionis Biosciences BV, Ghent (BE)

(72) Inventors: Nikolai KLEY, Waltham, MA (US); Samuel LIEVENS, Ghent (BE)

(21) Appl. No.: 17/785,676

(22) PCT Filed: Dec. 17, 2020
(86) PCT No.: PCT/US20/65463

§ 371 Date: Jun. 15, 2022

Related U.S. Application Data
(60) Provisional application No. 62/949,026, filed on Dec. 17, 2019.

(51) Int. Cl. G01N 33/68; C12N 15/10; C07K 14/715; C07K 14/71; C07K 14/72; C07K 14/705; C12N 9/10

(52) U.S. Cl. CPC G01N 33/6845; C12N 15/1055; C07K 14/715; C07K 14/71; C07K 14/72; C07K 14/70596; C12N 9/104; C12Y 203/02; C07K 2319/03; C07K 2319/033; C07K 2319/095; C07K 2319/61

(57) ABSTRACT

The present invention is related to a method for detecting and identifying protein-protein or protein-small molecule interactions using a bait and prey system. It is also related to bait and prey proteins, small molecules and constructs that are used for the methods described herein.

### CROSS-REFERENCE TO RELATED APPLICATIONS

This application claims the benefit of U.S. Provisional Application No. 62/949,026, filed on Dec. 17, 2019, the entire contents of which are incorporated herein.

### FIELD

The present invention is related to, inter alia, detection and identification protein-protein or protein-small molecule interactions, and/or novel small molecules.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “ORN-064PC_ST25.txt”; Date created: Dec. 7, 2020; File size: 10,365 bytes).

### BACKGROUND

Molecular interactions, such as protein/protein and protein/small molecule interactions, are a key part of many, if not all, biological processes. Enhancement or blockage of molecular interactions can be used as a therapeutic strategy; however, identification of clinically relevant molecular interactions is often problematic.

An increasing understanding of the role of protein-protein interactions (PPIs) has led to the exploration of agents that stabilize/induce interactions between proteins, rather than disrupting them or blocking their enzymatic activity. Molecular glues refer to small-molecule PPI stabilizers that bind a protein and modulate its molecular surface, thus enabling it to recruit a new protein, or stabilize a weak protein-protein interaction. These compounds, most notably, the immunomodulatory drug, lenalidomide, which interacts with the E3 ligase protein cereblon (CRBN) and drives downstream protein degradation, have demonstrated excellent efficacy in the treatment of various cancers. Certain molecular glues may also act by stabilizing weak protein-protein interactions by specifically binding to structures created at a protein-protein interaction interface. In this case, the molecular glue would bind only a configuration in which both proteins interact, versus scenarios outline herein in which a molecular glue, such as lenalidomide, binds to one protein first and then induces or enhances engagement of that complex with another protein(s). Thus, there remains a need for new more robust methods for detecting molecular interactions.

### SUMMARY

The present invention relates, in part, to a cell-based system for detecting various molecular interactions. In some embodiments, the present invention provides for methods that allow interrogation/identification of molecular interactions (e.g., protein/protein, protein/small molecule, and/or protein/protein interactions that are modulated by small molecules) which are not detectable using standard assays. In some embodiments, the methods disclosed herein allow for identification of clinically relevant or significant molecular interactions between proteins which can be exploited for development of a therapy against a disease.

In some embodiments, the methods disclosed herein include a bait protein and a plurality of prey proteins. Such methods may be used to identify molecular interactions between the bait protein and the plurality of prey proteins or an individual prey protein. In some embodiments, the present invention uses the mammalian protein-protein interaction trap assay (MAPPIT, see Eyckerman, et al. “Design and application of a cytokine-receptor-based interaction trap,” Nat Cell Biol. 2001 December; 3(12):1114-9 and Lievens, et al. “Proteome-scale Binary Interactomics in Human Cells,” Molecular & Cellular Proteomics 15.12 (2016): 3624-3639, incorporated by reference in their entireties). The MAPPIT-derived assays described herein, however, are enhanced by the use of a small molecule that interacts with the bait or the prey protein, as well as other features in some embodiments. In some embodiments, the molecular interactions between the bait protein and the prey protein are facilitated/induced by a small molecule (also described herein as “compound” or a “ligand” or a “drug”). In embodiments, the small molecule is a chemical entity that is not a hybrid ligand. In embodiments, the small molecule is a single chemical entity. In embodiments, the small molecule does not have a linker. In embodiments, the small molecule only directly interacts with one of the bait or prey protein. embodiments, the small molecule is a chemical entity that is a hybrid ligand, having one or more of: a CRBN-binding molecule, a PEG linker, and a small molecule.

In various embodiments, the present invention relates to a method for detecting a molecular interaction by (a) providing a cell having a ligand-based chimeric receptor comprising (i) an extracellular portion of a ligand-binding domain derived from a first receptor and (ii) transmembrane and cytoplasmic domains of the first receptor or a second receptor and having an intracellular E3 ligase substrate binding subunit bait protein fused thereto, wherein the transmembrane and/or cytoplasmic domains of the second receptor comprise mutations that reduce or eliminate STAT (Signal Transducer and Activator of Transcription) recruitment; (b) expressing a prey protein that is fused to a receptor fragment in the cell, the receptor fragment comprising functional STAT recruitment sites; and (c) detecting a signal that is indicative of a molecular interaction. In embodiments the binding of a small molecules to the E3 ligase substrate binding subunit promotes binding to the prey protein and formation of a protein complex that comprises the scaffold protein, the E3 ligase substrate binding subunit complexed with the small molecules, and the prey protein.

In some embodiments, the prey protein is fused to a receptor fragment. In some embodiments, the prey protein is fused to a receptor fragment, either N- or C-terminally. In embodiments, the prey protein is fused to gp130 or a fragment thereof. In embodiments, the prey protein is fused to gp130 or a fragment thereof either N- or C-terminally.

In embodiments, the first receptor and second receptor are the same.

In embodiments the E3 ligase substrate is endogenous or is expressed from a transgene.

In various embodiments, the present invention relates to a method for detecting a molecular interaction by (a) providing a cell having a ligand-based chimeric receptor comprising (i) an extracellular portion of a ligand-binding domain derived from a first receptor and (ii) transmembrane and cytoplasmic domains of the first receptor or a second receptor and having an scaffold protein fused thereto, wherein the transmembrane and/or cytoplasmic domains of the second receptor comprise mutations that reduce or eliminate STAT (Signal Transducer and Activator of Transcription) recruitment; (b) expressing a prey protein that is fused to a receptor fragment in the cell, the receptor fragment comprising functional STAT recruitment sites; and (c) detecting a signal that is indicative of a molecular interaction. In embodiments, the scaffold protein interacts with an E3 ligase substrate binding subunit and the complex of scaffold protein and E3 ligase substrate binding subunit interacts with the prey.

In some embodiments, the interaction between the prey protein and bait protein causes recruitment of the receptor fragment fused to the bait protein to the transmembrane chimeric receptor protein, which restores ligand-dependent transmembrane chimeric receptor signaling and activation of STAT molecules. In some embodiments, the cell comprises a STAT-responsive reporter gene. In some embodiments, the activated STAT molecules migrate to the nucleus and induce transcription of a STAT-responsive reporter gene and, in some instances, the reporter gene signal permits detection and/or discovery of a molecular interaction.

In some embodiments, the detected interaction is a recruitment of bait and/or prey into a binary, tertiary, or higher order protein complex.

In some embodiments, the molecular interaction is a protein/protein interaction. In some embodiments, the molecular interaction is a protein/protein interaction, which is mediated by a small molecule (e.g., the method further comprises introducing a small molecule which binds to the prey protein or bait protein). Specifically, in some embodiments, the molecular interaction is a protein/protein interaction, which is mediated by the binding of the small molecule with the prey protein or bait protein. For example, the present methods may detect a complex formation. In some embodiments, the small molecule induces exposure of a hydrophobic surface or a binding site of the prey protein or bait protein that allows for interaction with the prey protein or bait protein. In some embodiments, the small molecule is a molecular glue or a bivalent hybrid ligand molecule (e.g., without limitation a PROTAC).

By way of example, in some embodiments, the interactions detected involve an E3 ligase protein, e.g., without limitation, in contact with an Immunomodulatory Drug (IMiD) e.g., thalidomide, lenalidomide and pomalidomide, and compounds related thereto or compounds binding to an equivalent or similar structural pocket and small molecule binding site as normally occupied by IMiD compounds and compounds related thereto.

In some embodiments, the present methods are applicable to the use of VHL as a E3 ligase substrate binding bait protein. VHL is, similarly to CRBN, the substrate binding subunit of an E3 ligase. Accordingly all embodiments relating to an E3 ligase as bait are equally applicable to VHL as bait.

In embodiments, the present methods are applicable to the use FKBP12 protein or a member of this family, instead of an E3 ligase, as bait (accordingly all embodiments relating to E3 ligase as bait are equally applicable to an FKBP protein or a member of this family, e.g. without limitation, FKBP12, as bait).

In some embodiments, the present methods allow for display of a bait protein in which it is not expressed as a receptor-fusion protein. In this instance a different protein that can interact with the bait protein, namely a scaffold protein, is fused to the receptor protein. Interaction of the bait with such protein creates a protein complex that effectively displays the bait protein as part of the complex. This provides a new approach to display a bait protein in a form that does not require its fusion to a receptor. By way of example, and demonstrated herein, another component of an E3 ligase protein complex is fused to the receptor, such as, for example, DDB1 (or any other scaffold protein that naturally interacts with a substrate recognition component of an E3 ligase). As for DDB1, this protein, expressed as a receptor fusion, subsequently interacts with a CRBN bait protein expressed separately (e.g., as a non-fusion protein), displaying it in a context that mimics its natural form of presentation to substrates by an E3 ligase. Concomitant exposure to a molecular glue and a plurality of prey proteins enable the discovery of prey proteins that interact with the DDB1-CRBN complex in response to binding of CRBN to a molecular glue such as an MED. Similarly, by analogy, in some embodiments multiprotein complexes that comprise ligand-binding bait proteins other than CRBN, such as VHL or any other E3 ligase component, and that are not expressed receptor fusion proteins, can be displayed as baits in this fashion.

In some embodiments, the present methods are applicable to the screening of a plurality of prey proteins for interaction with bait and/or compound.

In various embodiments, the present methods pertain to an array-based format, e.g. in which cDNAs encoding various prey proteins are spotted on a surface. In various embodiments. the present methods pertain to a cell population-based method in which, e.g. a library of prey proteins is introduced into cells such that, on average, each cell expresses a single prey. In such embodiments, upon interaction with compound and/or bait, the encoding cDNA is identified to reveal the interactions. In embodiments, FACS or microfluidic separation is employed for the identification.

In some embodiments, the present methods are applicable to the screening of a plurality of compounds (e.g. a compound library) for interaction with prey proteins and/or bait proteins. In embodiments, in which the compound does not contain a linker (e.g. not a hybrid ligand), the present methods allow for screening without possible interference of compound interaction moieties due to linker attachment.

In some embodiments, the present methods are applicable to the use of VHL as a E3 ligase substrate binding bait protein. VHL is, similarly to CRBN, the substrate binding subunit of an E3 ligase (accordingly all embodiments relating to an E3 ligase as bait are equally applicable to VHL as bait).

In some embodiments the present methods are applicable to the use FKBP or a member of this family, instead of an E3 ligase, as bait that is not a receptor fusion (accordingly all embodiments relating to E3 ligase as bait that is not a receptor fusion are equally applicable to an FKBP or a member of this family, e.g. FKBP12, as bait).

### BRIEF DESCRIPTION OF THE DRAWINGS

FIGS. 1A-B shows non-limiting schematics that describes the present MAPPIT-derivative concept. In FIG. 1A, an E3 ligase substrate binding subunit bait protein (“B”) is C-terminally fused to a chimeric receptor. This chimeric receptor, e.g., has an extracellular part of a type I cytokine receptor (“CYT”) and transmembrane and intracellular domains of a receptor that is made deficient in STAT recruitment via mutagenesis. This chimeric receptor is signaling deficient. When co-expressed with a prey protein (“P”)—that is fused to a receptor fragment containing functional STAT recruitment sites—the receptor complex is functionally complemented, and, in some instances, upon cytokine ligand stimulation (L), STAT signaling is restored. STAT molecules are activated and migrate to the nucleus and induce transcription of a STAT-responsive reporter gene. In FIG. 1B is schematic that is similar to FIG. 1A but describes the present MAPPIT-derivative concept that involved displaying a bait protein in a form that is not a receptor fusion is shown. The cross-hatched segment is a scaffold protein.

FIGS. 2A-L Evaluation of CRBN-binding compounds recruiting selected substrates in a MAPPIT-derivative assay. Recruitment induced by the indicated CRBN IMiD ligands (thalidomide, THL; lenalidomide, LEN; pomalidomide, POM; CC-122; CC-220; CC-885) of a panel of CRBN substrates was evaluated in a MAPPIT-derivative assay. MAPPIT is a variation of a two-hybrid technology system that was described previously (Lemmens, et al. “MAPPIT, a mammalian two-hybrid method for in-cell detection of protein-protein interactions,” Methods Mol Biol. 2015; 1278:447-55, the entire contents of which are herein incorporated by reference) and is outlined in more detail in Example 1. The assay entails co-transfection of a CRBN bait receptor fusion together with a gp130-fused substrate fusion. Test compound activity was assessed with increasing concentrations of test compounds (dose-response studies) to monitor the ability to promote CRBN-ligand-induced protein interaction—i.e., recruitment of any of the indicated neosubstrates: IKZF1, GSPT1, GSPT2 and an undisclosed substrate. As shown, the results obtained reproduces literature known data generated with different technologies as described here. For example, any of the indicated compounds results in recruitment of IKZF1, whereas GSPT1 and GSPT2 are only recruited through CC-885.

FIGS. 3A-D Multiple CRBN MAPPIT-derivative receptor constructs enable the detection of compound-dependent substrate interactions. As discussed in more detail in Example 2, multiple receptor fusion configurations can be applied in the MAPPIT-derivative assay used here. A typical fusion protein consists of the extracellular domain of the EPO receptor fused to the transmembrane and intracellular portion of the mutated leptin receptor (FIGS. 3A-B). However, the extracellular EPO receptor domain can be exchanged for that of the leptin receptor, resulting in an assay system that is activated by leptin rather than EPO (FIGS. 3C-D). Similarly, alternative gp130 fusion proteins can be used where the partial gp130 domain can be fused either to the N- or C-terminus of the protein of interest. Here we tested CC-220- and CC-885-dependent CRBN interactions with IKZF1 and GSPT1 substrates applying multiple CRBN bait receptor fusion construct types and substrate gp130 fusions. Similar results were obtained with the EPO receptor-based CRBN receptor fusion (pSEL-CRBN) and the leptin receptor-based variant (pCLG-CRBN). Also, the different gp130 fusion versions, N- or C-terminal fusions, yield comparable data. In addition, we show that multiple versions of the substrate proteins, e.g. IKZF1 isoform 1 versus 7 or GSPT1 isoform 1 (full size) versus a partial construct covering only domain 2 and 3, yield similar results. In each set of histograms, the leftmost bar is 0 μM, the next bar to the right is 0.1 μM, the next bar to the right is 1 μM, and the rightmost bar is 10 μM.

FIG. 4 CRBN compound-dependent substrate interactions can be detected using an alternative MAPPIT-derivative assay configuration applying a DDB1 receptor fusion. An alternative CRBN substrate binding assay was tested where DDB1 was fused to the MAPPIT chimeric receptor construct (pSEL-DDB1) and an unfused CRBN bait protein was co-expressed along with the substrate gp130 fusion protein, either IKZF1 (gp130-IKZF1) or an undisclosed substrate protein (gp130-targetX). In the absence of CRBN co-expression (‘no CRBN’), no lenalidomide (LEN)-induced signal could be observed. However, when an unfused CRBN expression construct was co-transfected, a LEN-dependent signal was obtained for both the IKZF1 and target X interaction. In each set of histograms, the leftmost bar is 0 μM LEN, the next bar to the right is 0.1 μM LEN, the next bar to the right is 1 μM LEN, and the rightmost bar is 10 μM LEN.

FIG. 5 Co-expression of unfused DDB1 enhances the sensitivity of a MAPPIT-derivative compound-dependent CRBN substrate interaction assay. The effect was evaluated of co-transfecting an unfused DDB1 expression construct in a MAPPIT-derivative assay setup for molecular glue-induced CRBN-IKZF1 interactions. The assay configuration was similar as the one applied in FIGS. 2A-L, where a CRBN bait receptor construct (pSEL-CRBN) and an IKZF1 (isoform 7) gp130 fusion construct were co-expressed, without or with the additional DDB1 expression vector. A compound concentration-dependent induction of the reporter signal was observed for each of the tested molecular glues (same panel as used in FIGS. 2A-L), in either the absence or presence of DDB1 co-expression. Interestingly, signals were observed to be increased at the lower concentrations tested compared to the maximal signal in the setup where DDB1 was overexpressed, indicating that the sensitivity of the assay was higher than in the absence of DDB1 co-expression. In each set of histograms, the leftmost bar is 0 μM, the next bar to the right is 0.1 μM, the next bar to the right is 1 μM, and the rightmost bar is 10 μM.

FIG. 6A DDB1-CRBN MAPPIT-derivative receptor fusion enables detection of compound-dependent substrate recruitment. Here an assay configuration was tested where a DDB1-CRBN genetic fusion was tethered to the MAPPIT chimeric receptor construct (pSEL-DDB1-CRBN) and tested against a gp130-IKZF1 (isoform 7) substrate fusion in the presence of increasing concentration of a panel of molecular glues (same panel as used in FIGS. 2A-L). Also this assay configuration was able to reproduce compound-induced CRBN-IKZF1 complex formation in a compound dose-dependent manner. In each set of histograms, the leftmost bar is 0 μM, the next bar to the right is 0.1 μM, the next bar to the right is 1 μM, and the rightmost bar is 10 μM.

FIGS. 7A-B ARV-825 PROTAC-dependent recruitment of BRD4 substrate to CRBN can be detected in MAPPIT. The alternative MAPPIT-derivative CRBN bait receptor fusions tested in FIGS. 3A-D, either containing the EPO receptor extracellular domain (pSEL-CRBN) or the leptin receptor extracellular domain (pCLG-CRBN), were combined with N- or C-terminal gp130 fusions of BRD4 (isoform 3). In both assays, the ARV-825 PROTAC (a chemical fusion of a CRBN binding ligand and a BRD4 binding compound) induced a dose-dependent luciferase reporter signal. In each set of histograms, from left to right, the bars represent the following: 0 μM ARV-825; 0.0003 μM ARV-825; 0.003 μM ARV-825; 0.03 μM ARV-825; 0.3 μM ARV-825; 3 μM ARV-825; and 30 μM ARV-825.

FIGS. 8A-F MAPPIT-derivative assay enables detection of compound-dependent interactions between FKBP1A (FKBP12) and MTOR or calcineurin. Compound-dependent FKBP1A interactions with known target proteins were evaluated using an FKBP1A bait receptor fusion (pSEL-FKBP1A) combined with MTOR (FRB domain) or calcineurin PPP3CA catalytic subunit gp130 fusion proteins. As shown, compound-induced recruitment of MTOR is detected with both rapamycin and everolimus. Similarly, also FK506- or pimecrolimus-dependent binding of PPP3CA can be monitored. Of note, in the case of calcineurin binding, co-expression of the PPP3R2 regulatory subunit increases the signal window significantly.

FIGS. 9A-B Hybrid ligand-induced BRD4 substrate recruitment to VHL can be detected in MAPPIT. Here, VHL bait protein was fused to a MAPPIT-derivative chimeric receptor construct containing the EPO receptor extracellular domain (pSEL-VHL) or the leptin receptor extracellular domain (pCLG-VHL). These constructs were combined with the same N- or C-terminal gp130 fusions of BRD4 (isoform 3) used in FIG. 7, or an unfused gp130 negative control construct. Cells expressing both the VHL and BRD4 constructs were treated with a concentration range of MZ1 (a chemical fusion of a VHL and a BRD4 ligand), inducing a dose-dependent MAPPIT signal. No signal was obtained when the unfused gp130 control construct was tested. In each set of histograms, the leftmost bar is 0 μM MZ1, the next bar to the right is 0.1 μM MZ1, the next bar to the right is 1 μM MZ1, and the rightmost bar is 10 μM MZ1.

FIGS. 10A-C Screening of a compound collection identifies novel molecular glues that enable recruitment of IKZF1 to CRBN. The MAPPIT-derivative assay applied in FIGS. 2A-L, where a CRBN bait receptor construct (pSEL-CRBN) and an IKZF1 (isoform 7) gp130 fusion construct were co-expressed, was used to screen a collection of 96 IMiDs and IMiD-like compounds. In a primary screen, the compounds were tested at 3 doses (low, medium and high concentration) and luciferase reporter signal was determined. The curves shown in FIGS. 10A-C represent luciferase signal frequency distributions for both compound-treated samples and DMSO-treated controls (left panel). The curve for the compound-treated samples is bimodal, where the right-shifted peak covers compounds that exhibit a reporter signal that is higher than that for the DMSO-treated controls. For three compounds exhibiting a response and thus representing compounds that induce recruitment of IKZF1 to CRBN the dose-response hit confirmation is shown (right panel). The corresponding signal at each of the tested concentrations in the primary screen is indicated by line marks with a dash type corresponding to the one used in the dose-response curves (dotted, dashed or solid). These sample curves indicate that this approach is able to identify molecular glues across a broad potency range.

FIGS. 11A-B ORF cDNA library screening detects novel molecular glue-induced CRBN neosubstrates. Here, a MAPPIT-derivative assay was applied in a cell microarray-based screening format to screen a human ORF(eome) cDNA library for targets recruited to CRBN in response to CC-220, a known MED drug and CRBN ligand. Protein and small molecule interactions in cells were assayed within cell clusters displayed in an array format. Each spot in a cell microarray corresponded to such a cell cluster expressing a single ORF/protein candidate that is being tested for ligand-induced (in this case CC-220-induced) interaction with CRBN. A positive interaction was read out as an increase in cell fluorescence. Shown is a dot plot of the fluorescence intensity data from a cell microarray screen across/for a large number of individual ORFs/target protein candidates. The X-axis shows the Particle Count and the Y-Axis shows the integral intensity for each cell cluster in the microarray. As shown, and indicated, a significant induction of signal is observed for a number of ORF cDNAs. For four ORF cDNAs exhibiting a response and therefore representing proteins being recruited to CRBN through the CC-220 molecular glue (indicated by arrows), dose-response curves were generated to confirm their CC-220 dose-dependent binding to CRBN. These examples show that this MAPPIT-derivative screening approach enables identifying novel molecular glue-induced substrates of CRBN.

FIGS. 12A-B Hybrid ligand compound screening identifies known and novel ligands of a target protein of interest. A MAPPIT-derivative assay was used to screen a collection of trimethoprim (TMP)-fused hybrid ligand molecules for binding to a target protein of interest. In this assay, advantage is taken of the high affinity of TMP for DHFR to anchor the TMP hybrid ligands to the DHFR receptor fusion and display the TMP-linked compound as a bait. In FIG. 12A, a MAPPIT-derivative assay where a DHFR receptor fusion (in this case a receptor fusion protein containing the extracellular domain of the leptin receptor; pCLG-DHFR) was co-expressed with a gp130 fusion of the estrogen receptor (ESR1), was used to screen a 320 member hybrid ligand diversity set (containing compounds from a diversity collection, each linked to TMP through a PEG linker) spiked with a TMP fusion of tamoxifen (TAM), a known ligand of ESR1. The compounds were screened at a single dose and luciferase reporter signal was determined. The curve shown in FIG. 12A represents the luciferase signal frequency distribution for both compound-treated samples and DMSO-treated controls (left panel). As expected for a diversity set, both distributions largely overlap, except for a small number of compounds for which the compound-treated signal is higher than the DMSO control signal. One of these hits corresponded with TMP-TAM (solid line mark on the frequency curve). Dose-response analysis confirmed the signal obtained for TMP-TAM binding to ESR1, with an EC50 in the low nanomolar range, as reported in the literature. A similar screening setup was applied to identify novel ligands of MDM4, a validated cancer target protein. A single hit was identified in the hybrid ligand screen represented in FIG. 12B, which could be confirmed in dose-response follow-up experiments. These examples show that the MAPPIT-derivative assay applied here can be used to identify novel ligands of a particular target protein of interest.

FIGS. 13A-B Identification of novel hybrid ligand targets through array-based ORF cDNA library screening. Here, a cell microarray-based screening approach as used in FIGS. 11A-B was applied to screen a human ORF(eome) cDNA library for targets of TMP-fused hybrid ligands, using a MAPPIT-derivative assay as described in FIGS. 12A-B. The compound of interest, in this case an undisclosed compound with a strong antitumor phenotype for which the target was not known, was displayed as a TMP hybrid ligand bait anchored to the DHFR receptor fusion and interaction with any of the proteins encoded by the arrayed ORF cDNA gp130 fusions is detected as increased cell fluorescence of the corresponding spot on the array. The dot plot shown represents the fluorescence data from a cell microarray screen across/for a large number of individual ORFs/target protein candidates. The X-axis shows the particle count and the Y-Axis shows the integral intensity for each cell cluster in the microarray. As shown, a strong signal was observed for a specific ORF cDNA (indicated by an arrow), and this interaction could be confirmed in dose-response analysis. This data illustrates that the MAPPIT-derivative screening approach described here enables the identification of novel targets for ligands through the use of TMP-derivatized ligand fusion molecules.

FIG. 14 Identification of rapamycin-induced binding between FKBP proteins and MTOR. Different members of the FKBP protein family (FKBP1A/FKBP12, FKBP3, FKBP4 and FKBP5) were evaluated in a MAPPIT-derivative assay for recruitment of MTOR (FRB domain), where the FKBP protein was expressed as a MAPPIT receptor fusion containing the Epo receptor extracellular domain (pSEL-FKBPx) and MTOR(FRB) was fused to gp130. As shown, for each of the tested FKBP proteins a rapamycin dose-dependent signal was obtained, in line with published reports. In each set of histograms, the leftmost bar is 0 nM rapamycin, the next bar to the right is 1 nM rapamycin, the next bar to the right is 10 nM rapamycin, and the rightmost bar is 100 nM rapamycin.

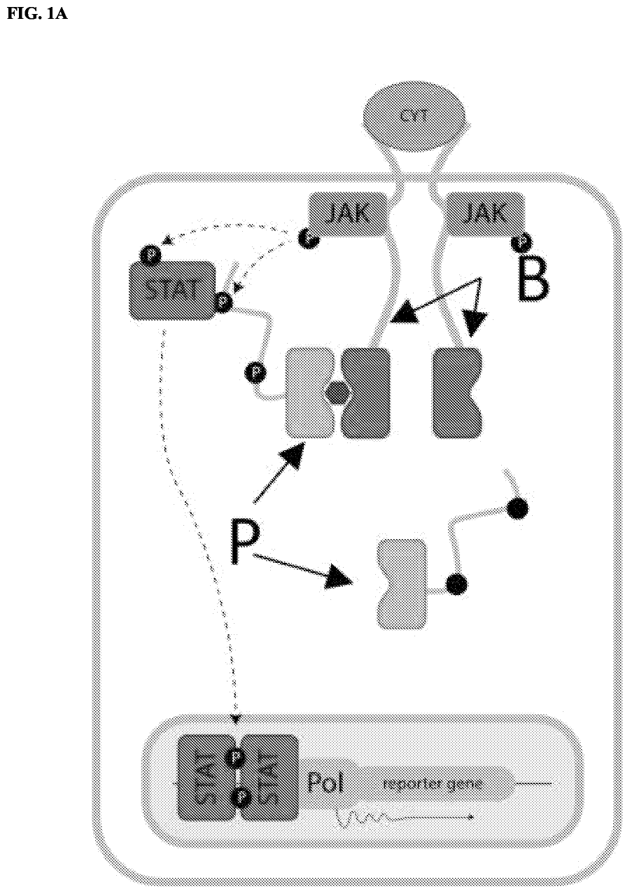

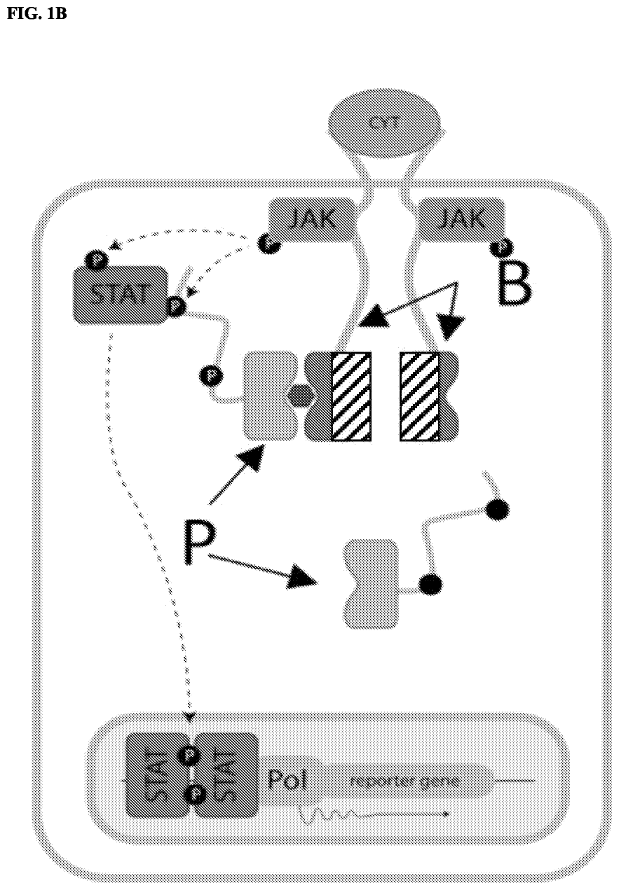

### DETAILED DESCRIPTION

The present disclosure is based, in part, on the discovery of cell-based systems and methods that allow interrogation of molecular interactions (e.g., protein/protein, protein/small molecule, and/or protein/protein interactions that are modulated by small molecules) which are not detectable using standard assays.

In one aspect, the present methods allow for a method for detecting a molecular interaction, comprising: (a) providing a cell comprising a ligand-based chimeric receptor comprising (i) an extracellular portion of a ligand-binding domain derived from a first receptor and (ii) transmembrane and cytoplasmic domains of such first receptor or a second receptor, and having an intracellular E3 ligase substrate binding subunit bait protein fused thereto, wherein the transmembrane and/or cytoplasmic domains of the receptor construct comprise mutations that reduce or eliminate STAT recruitment; (b) expressing a prey protein that is fused to a receptor fragment in the cell, the receptor fragment comprising functional STAT recruitment sites; and (c) detecting a signal that is indicative of a molecular interaction.

In various embodiments, the present invention relates to a method for detecting a molecular interaction by (a) providing a cell having a ligand-based chimeric receptor comprising (i) an extracellular portion of a ligand-binding domain derived from a first receptor and (ii) transmembrane and cytoplasmic domains of the first receptor or a second receptor and having an scaffold protein fused thereto, wherein the transmembrane and/or cytoplasmic domains of the receptor construct comprise mutations that reduce or eliminate STAT (Signal Transducer and Activator of Transcription) recruitment; (b) expressing a prey protein that is fused to a receptor fragment in the cell, the receptor fragment comprising functional STAT recruitment sites; and (c) detecting a signal that is indicative of a molecular interaction. In embodiments, the scaffold protein interacts with an E3 ligase substrate binding subunit and the complex of scaffold protein and E3 ligase substrate binding subunit interacts with the prey.

In some aspects, the present methods allow for a method for detecting a molecular interaction, comprising: (a) providing a cell comprising a ligand-based chimeric receptor comprising (i) an extracellular portion of a ligand-binding domain derived from a first receptor and (ii) transmembrane and cytoplasmic domains of such first receptor or a second receptor, and having a protein (or more) fused thereto and that can interact with an intracellular E3 ligase substrate binding subunit bait protein, wherein the transmembrane and/or cytoplasmic domains of the receptor construct comprise mutations that reduce or eliminate STAT recruitment; (b) expressing a prey protein that is fused to a receptor fragment in the cell, the receptor fragment comprising functional STAT recruitment sites; and (c) detecting a signal that is indicative of a molecular interaction. In embodiments, the bait protein is associated with a scaffold protein and an E3 ligase substrate binding subunit bait. In embodiments, the bait protein is directly fused to the transmembrane protein.

In some embodiments, the interaction between the prey protein and bait protein in systems such as described herein causes formation of a protein complex which comprises the receptor fragment fused to the prey protein. Recruitment of such receptor fragment into the complex, thereby positioning of it as an available substrate for a receptor-associated JAK kinase (e.g., JAK2), restores ligand-dependent receptor signaling and activation of STAT molecules. In some embodiments, the cell comprises a STAT-responsive reporter gene. In some embodiments, and the activated STAT molecules migrate to the nucleus and induce transcription of a STAT-responsive reporter gene and, in some instances, the reporter gene signal permits detection and/or discovery of a molecular interaction.

In some embodiments, the molecular interaction is a protein/protein interaction. In some embodiments, the bait and prey are both proteins.

The present invention also includes analyzing a library of compounds. In embodiments, the bait binds to the compound and, optionally this bait-compound complex interacts with the prey. In embodiments, therefore, the present methods allow for the detection and/or discovery of novel compound mediated protein/protein interactions and/or novel protein/compound interactions. In embodiments, the present methods allow for the detection and/or discovery of novel compounds that act as molecular glues. In embodiments, the present methods allow for the detection and/or discovery of novel compound which converts a weak bait-prey interaction into a stronger bait-prey interaction.

In some embodiments, the bait is or comprises a protein that modulates the ubiquitin-proteasome system. In some embodiments, the bait is or comprises an E3 ligase protein, or a protein that modulates an E3 ligase protein. In some embodiments, the bait is or comprises a cullin-RING ligase (CRL) protein, or a protein that modulates an CRL protein. In various embodiments, the bait is or comprises a CRL4 protein, or a protein that modulates an CRL4 protein. In some embodiments, the bait is or comprises a DDB1-CUL4-associated factor (DCAF) protein, or a protein that modulates a DCAF.

In some embodiments, the bait is or comprises one or more of cereblon (CRBN) and Von Hippel Lindau (VHL).

In embodiments, the CRBN or VHL is fused to the transmembrane domain as described herein. In embodiments, the CRBN or VHL is not fused to the transmembrane domain as described herein, e.g., it operates as a bait upon interacting with a scaffold protein, which is fused to the transmembrane domain as described herein.

In embodiments, the bait is an E3 ligase substrate binding subunit.

In embodiments, the E3 ligase substrate binding subunit is selected from the protein encoded by any of the following genes: AMFR, ANAPC11, APG16L, ARIH1, ARIH2, ARPC1A, ARPC1B, ASB2, ASB2, ATG16L1, BAF250, BARD1, BIRC2, BIRC3, BIRC4, BIRC7, BMI1, BRAP, BRCA1, bTrCP, CBL, CBLB, CBLC, CBLL1, CCIN, CCIN, CCNB1IP1, CRBN, CHFR, CHIP, CNOT4, COP1, CSA, DCAF1, DCAF10, DCAF11, DCAF12, DCAF13, DCAF14, DCAF15, DCAF16, DCAF17, DCAF19, DCAF2, DCAF3, DCAF4, DCAF5, DCAF6, DCAF7, DCAF8, DCAF9, Ddal, DDB2, DET1, DNAI2, DTX3, DZIP3, E6AP, EDD, EED, ENC1, ENC1, FANCL, FBXL1, FBXL10, FBXL11, FBXL12, FBXL13, FBXL14, FBXL15, FBXL16, FBXL17, FBXL18, FBXL19, FBXL20, FBXL21, FBXL22, FBXL3, FBXL4, FBXL5, FBXL7, FBXL8, FBXO1, FBXO10, FBXO11, FBXO12, FBXO13, FBXO14, FBXO15, FBXO16, FBXO17, FBXO18, FBXO19, FBXO2, FBXO20, FBXO21, FBXO22, FBXO3, FBXO4, FBXO5, FBXO6, FBXO7, FBXO8, FBXW1, FBXW10, FBXW11, FBXW12, FBXWS, FBXW7, FBXW8, FBXW9, FEM1A, FEM1B, FEM1C, GAN, GAN, GNB1, GNB2, GNBS, GRWD1, GTF2H2, GTF3C2, HACE1, HECTD1, HECTD2, HECTD3, HERC1, HERC2, HERC3, HERC4, HERC5, HERC6, HLTF, HOIP, HUWE1, IBRDC2, IBRDC3, IFRG15, IPP, IPP, ITCH, IVNS1ABP, IVNS1ABP, KATNB1, KBTBD10, KBTBD10, KBTBD11, KBTBD11, KBTBD12, KBTBD12, KBTBD13, KBTBD13, KBTBD2, KBTBD2, KBTBD3, KBTBD3, KBTBD4, KBTBD4, KBTBD5, KBTBD5, KBTBD6, KBTBD6, KBTBD7, KBTBD7, KBTBD8, KBTBD8, KCTD5, KEAP, KEAP1, KIAA0317, KIAA0614, KLHDC5, KLHL1, KLHL1, KLHL10, KLHL10, KLHL11, KLHL11, KLHL12, KLHL12, KLHL13, KLHL13, KLHL14, KLHL14, KLHL15, KLHL15, KLHL17, KLHL17, KLHL18, KLHL18, KLHL2, KLHL2, KLHL20, KLHL21, KLHL21, KLHL22, KLHL22, KLHL23, KLHL23, KLHL24, KLHL24, KLHL25, KLHL25, KLHL26, KLHL26, KLHL28, KLHL28, KLHL29, KLHL29, KLHL3, KLHL3, KLHL30, KLHL30, KLHL31, KLHL31, KLHL32, KLHL32, KLHL33, KLHL33, KLHL34, KLHL34, KLHL35, KLHL35, KLHL36, KLHL36, KLHL38, KLHL38, KLHL4, KLHL4, KLHL5, KLHL5, KLHL6, KLHL6, KLHL7, KLHL7, KLHL8, KLHL8, KLHL9, KLHL9, LINCR, LNX1, LRR1, LRRC41, LRSAM1, LZTR1, LZTR1, MAGEA1, MAGE-A1, MAGEA2, MAGE-A2, MAGEA3, MAGE-A3, MAGEA6, MAGE-A6, MAGEB18, MAGE-B18, MAGEB2, MAGE-B2, MAGEC2, MAGE-C2, MALIN, MAP3K1, MARCH1, MARCH11, MARCH2, MARCH4, MARCH5, MARCH6, MARCH7, MARCH8, MARCH9, MDM2, MDM4, MEX, MGRN1, MIB1, MIB2, MID1, MKRN1, MNAT1, MUF1, MULAN, MURF, MYCBP2, MYLIP, Nedd4, NEDD4L, NEDL1, NEDL2, NEURL, NEURL2, NLE1, NUP43, OSTM1, PAFAH1B1, PARC, PARK2, PCGF1, PCGF2, PDZRN3, PEX10, PEX7, PJA1, PJA2, POC1A, PPIL2, PRAME, PRPF19, PWP1, RACK1, RAD18, RAE1, RAG1, RBBP4, RBBP5, RBBP6, RBBP7, RBCK1, RBX1, RCHY1, RFFL, RFPL4A, RFWD2, RING1, RNF103, RNF11, RNF111, RNF114, RNF12, RNF123, RNF125, RNF128, RNF13, RNF130, RNF133, RNF135, RNF138, RNF139, RNF14, RNF144A, RNF167, RNF168, RNF180, RNF181, RNF182, RNF185, RNF19, RNF2, RNF20, RNF20, RNF216, RNF25, RNF34, RNF4, RNF40, RNF41, RNF43, RNF43, RNF5, RNF6, RNF7, RNF8, RNF85, RPTOR, SCAP, SH3RF1, SHPRH, SIAH1, SIAH2, SMU1, SMURF1, SMURF2, SOCS1, SOCS3, SPOP, SPSB1, SPSB1, SPSB2, SPSB2, SPSB4, SPSB4, STXBP5L, SYVN1, TAFSL, TBL1Y, THOC3, TLE1, TLE2, TLE3, TOPORS, TRAF2, TRAF6, TRAF7, TRAIP, TRIAD3, TRIM1, TRIM10, TRIM11, TRIM12, TRIM13, TRIM14, TRIM15, TRIM16, TRIM17, TRIM18, TRIM2, TRIM21, TRIM22, TRIM23, TRIM24, TRIM25, TRIM26, TRIM27, TRIM28, TRIM29, TRIM29, TRIM3, TRIM31, TRIM32, TRIM33, TRIM36, TRIM37, TRIM39, TRIM40, TRIM41, TRIM44, TRIM45, TRIM47, TRIMS, TRIM50, TRIM52, TRIM54, TRIM55, TRIM58, TRIM59, TRIM62, TRIM65, TRIM66, TRIM7, TRIM71, TRIM8, TRIM9, TRIP12, TRPC4AP, TSSC1, UBE3B, UBE3C, UBE4A, UBE4B, UBOX5, UBR1, UBR2, UBR3, UBR4, UHRF1, UHRF′2, VHL, VPS18, WDR12, WDR23, WDR26, WDR3, WDR31, WDR37, WDR39, WDR4, WDR47, WDR48, WDR5, WDR51B, WDR53, WDR57, WDR59, WDR5B, WDR61, WDR76, WDR77, WDR82, WDR83, WDR86, WSB1, WSB2, WWP1, WWP2, ZNF294, ZNF313, ZNF364, ZNRF1, ZNRF2, ZYG11A, ZYG11B, or ZYG11BL.

In embodiments, the E3 ligase substrate binding subunit is CRBN or VHL.

In embodiments, the scaffold protein interacts with an E3 ligase substrate binding subunit and the complex of scaffold protein and E3 ligase substrate binding subunit interacts with the prey.

In embodiments, the scaffold is selected from BIRC6, CUL3, DDB1, ELOB, ELOC, RBX1, SKP1, UBCH5A, UBE2A, UBE2B, UBE2B2, UBE2C, UBE2D1, UBE2D2, UBE2D3, UBE2D4, UBE2E1, UBE2E2, UBE2E3, UBE2F, UBE2G1, UBE2G2, UBE2H, UBE2J1, UBE2J2, UBE2K, UBE2L3, UBE2L6, UBE2M, UBE2N, UBE2NL, UBE2O, UBE2Q1, UBE2Q2, UBE2QL, UBE2R1, UBE2R2, UBE2S, UBE2T, UBE2U, UBE2V1, UBE2V1, UBE2V2, and UBE2W.

In some embodiments, the scaffold protein is selected from damaged DNA binding protein 1 (DDB1), Cullin-4A (CUL4A), and regulator of cullins 1 (ROC1).

In various embodiments, the bait comprises one or more of cereblon (CRBN), damaged DNA binding protein 1 (DDB1), Cullin-4A (CUL4A), regulator of cullins 1 (ROC1), and Von Hippel Lindau (VHL).

In some embodiments, the prey is a substrate and/or neosubstrate of CRBN. In embodiments, the substrate and/or neosubstrate of CRBN comprises b-hairpin a-turn with an i-residue bearing a side chain with a hydrogen bond acceptor, such as Asx or ST motifs, with a hydrogen bond between the sidechain of i and the backbone NH of i+3 and between the backbone carbonyl oxygen of i and the backbone NH of i+4. In embodiments, the i+4 residue is glycine (non-limiting examples include GSPT1, CK1a). In embodiments, the substrate and/or neosubstrate of CRBN has a b-hairpin a-turn with residues i and i+3 being cysteine and the i+4 residue being glycine. The two Cys residues bind to a zinc ion to enforce the shape of the turn (non-limiting examples include IKZF1, ZnF692 and all the substrate reported in “Defining the human C2H2 zinc finger degrome targeted by thalidomide analogs through CRBN”, Sievers et al, Science Vol. 362, Issue 6414, DOI: 10.1126/science.aat0572 (2018), incorporated by reference in its entirety). In embodiments, the substrate and/or neosubstrate of CRBN has a “pseudo-loop”, a b-hairpin b-turn bearing a glycine in the i+3 position. Turn structure can be enforced by a hydrogen bond between a hydrogen bond acceptor of the i−1 side chain and the carbonyl of the i+3 glycine.

In embodiments, CRBN refers to the polypeptides comprising the amino acid sequence of any CRBN, such as a human CRBN protein (e.g., human CRBN isoform 1 (GenBank Accession No. NP_057386); or human CRBN isoforms 2 (GenBank Accession No. NP_001166953), each of which is herein incorporated by reference in its entirety), and related polypeptides, including SNP variants thereof. Related CRBN polypeptides include allelic variants (e.g., SNP variants); splice variants; fragments; derivatives; substitution, deletion, and insertion variants; fusion polypeptides; and interspecies homologs, which, in certain embodiments, retain CRBN activity and/or are sufficient to generate an anti-CRBN immune response.

In some embodiments, the prey is one or more of Ikaros (IKZF1), Helios (IKZF2), Aiolos (IKZF3), Eos (IKZF4), Pegasus (IKZF5), SALL4, CSNK1A, CK1a, and ZFP91. In various embodiments, the prey is one or more of Ikaros (IKZF1), Helios (IKZF2), Aiolos (IKZF3), Eos (IKZF4), Pegasus (IKZF5), SALL4, CSNK1A, CK1a, and ZFP91. In some embodiments, the prey is one or more of Ikaros (IKZF1), Helios (IKZF2), Aiolos (IKZF3), Eos (IKZF4), Pegasus (IKZF5), SALL4, CSNK1A, CK1a, and ZFP91.

In some embodiments, the present method involves one or more E3 ligase substrate binding subunit, such as, without limitation, CRBN and VHL, as bait (or a bait associated with a scaffold protein, such as DDB1, CUL4A, and ROC1, in contact with CRBN or VHL), and the bait is contacted with a compound described herein (e.g., a compound that binds to one or more E3 ligase substrate binding subunit, such as, without limitation, CRBN and VHL, e.g., an MED) to discover a protein prey that interacts with the bait, as it is modulated by the compound. For example, the method identifies an interacting prey that contacts the bait, the bait being modified by the compound. In some embodiments, the prey is recruited and/or degraded because of the interaction with bait. In such embodiments, without limitation, the prey does not directly interact with the compound.

In some embodiments, the present methods allow for the identification of new substrates or neosubstrates of CRBN.

In some embodiments, the present method(s) involves one or more E3 ligase substrate binding subunit, such as, without limitation, CRBN and VHL as bait (or a bait associated with a scaffold protein, such as DDB1, CUL4A, and ROC1, in contact with CRBN or VHL), and the bait is contacted with a test compound in the presence of a protein prey that interacts with the bait (e.g., without limitation a substrate or neosubstrate of the E3 ligase substrate binding subunit), to detect a new small molecule-modulated protein/protein interaction. For example, the method identifies a compound as one that is capable of interacting with an E3 ligase substrate binding subunit bait and, in complex with such bait, with a substrate or neosubstrate of the E3 ligase substrate binding subunit. For example, the method identifies a compound as one that is capable of interacting with an E3 ligase substrate binding subunit and modulating the recruitment and/or ubiquitination and/or degradation of a second protein (e.g. the prey, e.g., without limitation a substrate or neosubstrate of the E3 ligase substrate binding subunit).

In some embodiments, the bait protein of the present invention is an E3 ligase substrate binding subunit. E3 ligases (also called ubiquitin ligase) are a class of diverse proteins, and functionally they recognize a target protein and mediate covalent linkage between target protein and ubiquitin moieties. They provide target specificity and uniqueness in the process of ubiquitination. E3 ligase recruits E2 ubiquitin-conjugating enzyme that has been loaded with ubiquitin, recognizes a target protein, and assists or directly catalyzes the transfer of ubiquitin from the E2 to the protein substrate.

The methods described in the present invention can be performed using any of the E3 ligases known in the art. In some embodiments, E3 ligases of the present invention include a protein that interacts with both E2-ubiquitin thioester and the substrate protein and catalyzes efficient ubiquitin transfer to the lysine residue of the target protein (polyubiquitin chain initiation) or ubiquitin in a growing chain. In some embodiments, the methods of the present invention include a subunit of the E3 ligase. The E3 ligase subunit according to the present invention can be a functional E3 ligase or a non-functional portion of a functional E3 ligase.

In some embodiments, the E3 ligase, or a subunit thereof, of the present invention is selected from cereblon (CRBN) and Von Hippel Lindau (VHL).

In one embodiment, the E3 ligase of the present invention is cereblon or a subunit thereof.

In some embodiments, the scaffold protein is damaged DNA binding protein 1 (DDB1), Cullin-4A (CUL4A), regulator of cullins 1 (ROC1), SKP1, SKP1 interacting partner (SKIP2), Beta-transducin repeats-containing protein (β-TrCP), Double minute 4 protein (MDM4), X-Linked Inhibitor of Apoptosis (XIAP), DDB1 And CUL4 Associated Factor 15 (DCAF15), and WD Repeat Domain 12 (WDR12) or subunits thereof.

In some embodiments, the present methods allow for the identification of new interaction partners, e.g., substrates or neosubstrates of a protein that binds to a compound, the protein having a cage of three tryptophan residues that are capable of interacting with a glutarimide ring of the compound, e.g., via hydrogen binding. In some embodiments, the interaction partner, e.g., substrate and/or neosubstrate, has a surface β-hairpin loop, the surface β-hairpin loop optionally having an arrangement of three backbone hydrogen bond acceptors at the apex of a turn followed by a glycine residue. In some embodiments, the interaction partner, e.g., substrate and/or neosubstrate, has a degron motif (see, Meszaros, et al. Sci Signal 2017: 10, 470, the entire contents of which are incorporated by reference).

In some embodiments, the bait is a protein having a cage of three tryptophan residues that are capable of interacting with a glutarimide ring of the compound (such as, immunomodulatory drugs or immunomodulatory imide drugs (IMiDs)), e.g., via hydrogen binding.

In some embodiments, the prey, e.g., substrate and/or neosubstrate, has a surface β-hairpin loop, the surface β-hairpin loop optionally having an arrangement of three backbone hydrogen bond acceptors at the apex of a turn followed by a glycine residue. In some embodiments, the prey, e.g., substrate and/or neosubstrate, has a degron motif (see, Meszaros, et al. Sci Signal 2017: 10, 470, the entire contents of which are incorporated by reference).

In various embodiments, the disclosed methods identify a protein/protein interaction which is mediated by the binding of a small molecule with the prey protein or bait. In some embodiments, the method further comprises introducing a small molecule which binds to the prey protein or bait protein. In some embodiments, the molecular interaction is a protein/protein interaction which is mediated by the binding of the small molecule with the prey protein or bait protein.

In some embodiments, the molecular interaction is two or more protein/protein interactions which are mediated by the binding of the small molecule with the prey protein or bait protein. In one embodiment, the small molecule binds to the bait protein and this binding causes a change in the bait protein such that it—after binding with the small molecule—is capable of binding to the prey protein. For example, in one embodiment, the binding of the small molecule to the bait protein causes a conformational change in the bait protein, e.g., a binding site on the bait protein may become accessible for the prey protein to bind to the bait protein. In another embodiment, binding of the small molecule to the bait protein opens up or exposes a hydrophobic binding site within the bait protein such that the prey protein can bind to the hydrophobic binding site of the bait protein.

In other embodiments, the small molecule binds to the prey protein and this binding causes a change in the prey protein such that it can now interact/bind with the bait protein. In some embodiments, the binding of the small molecule to the prey protein causes a conformation change in the prey protein such that a binding site on the prey protein becomes accessible to the bait protein so it can bind to the prey protein. In other embodiments, binding of the small molecule to the prey protein opens up or exposes a hydrophobic binding site within the prey protein such that the bait protein can interact with the hydrophobic binding site of the prey protein.

In yet other embodiments, the present method include small molecules that do not bind to the bait protein or the prey protein; but bind to the complex between the bait protein and the prey protein. For example, the interaction between bait protein and the prey protein could reorganize or create a binding site for the small molecule. In some embodiments, the small molecule binding site is present in the bait protein and exposed upon complex formation between the bait protein and the prey protein. In other embodiments, the small molecule binding site is present in the prey protein and exposed upon complex formation between the bait protein and the prey protein. In some embodiments, the interaction between the bait protein and the prey protein exposes an existing small molecule binding site or induces the formation of a small molecule binding site.

In some embodiments, the protein/protein interaction which is mediated by the binding of the small molecule with the prey protein or bait protein is a direct binding between the prey protein or bait protein and the small molecule at a protein/protein interface or within the protein. For example, in one embodiment, the small molecule can bind to the bait protein directly forming a bait protein-small molecule complex. In another embodiment, the small molecule can bind to the prey protein directly forming a prey protein-small molecule complex.

The present invention also envisions molecular interactions where the small molecule, the prey and the bait proteins interact with each other simultaneously. For example, in one embodiment, the small molecule binds directly to both the bait protein and the prey protein. In some embodiments, the protein/protein interaction which is mediated by the binding of the small molecule with the prey protein or bait protein is mediated by an allosteric modification of the protein surface of the prey protein or bait protein. In some embodiments, the protein/protein interaction which is mediated by the binding of the small molecule with bait protein is mediated by an allosteric modification of the protein surface of the bait protein.

In some embodiments, the small molecule induces exposure of a hydrophobic surface of the bait protein that allows for interaction with the prey protein. In some embodiments, the small molecule induces exposure of a hydrophobic surface of the prey protein that allows for interaction with the bait protein.

In some embodiments, the small molecule is a molecular glue. Molecular glues are molecules that promote, in some instances, the unnatural association of proteins to produce a therapeutic effect. In some embodiments, the molecular glue is a molecule where two small molecules are linked together by a linker. For instance, in embodiments, the present compound is a hybrid ligand with a compound with interacts with one of CRBN, VHL, and FKBP.

In other embodiments, the molecular glue is one small molecule without any linkers connecting the small molecule to another small molecule. In some embodiments, the molecular interaction is a complex formation. In some embodiments, the molecular interaction is a small molecule/protein interaction.

In some embodiments, the small molecule or the compound is an immunomodulatory agent. In some embodiments, the compound is a derivative of glutamic acid that comprises a glutarimide ring, optionally, and a phthalimide ring. In some embodiments, the phthalimide ring is chemically modified. In some embodiments, the derivative of glutamic acid can be a synthetic derivative having the properties in accordance with embodiments of the present disclosure. In some embodiments, the compound is a member of the class of compounds known as immunomodulatory drugs or immunomodulatory imide drugs (IMiDs). In embodiments, the compound contains a IMiD-like glutarimide ring, but otherwise differs in chemical structure and binds to the same small molecule binding pocket as a glutaramide-IMiD in CRBN (the MED binding pocket in CRBN).

In embodiments, the compound does not contain a glutaramide ring and can bind CRBN in the IMiD pocket. In embodiments, the compound binds CRBN, but not in the IMiD pocket. In embodiments, the IMiD pocket is contained within the CULT (cereblon domain of unknown activity, binding cellular ligands and thalidomide) domain of CRBN, see PDB entries 4TZ4, 5FQD, 5HXB, 5V3O, 6H0F, and 6H0G and PLoS Comput Biol. 2015 January; 11(1): e1004023, each of which are incorporated by reference in their entireties.

In some embodiments, the compound is thalidomide, lenalidomide, pomalidomide, CC-220, CC-122, CC-885, or a derivative, analog, enantiomer or a mixture of enantiomers, or a pharmaceutically acceptable salt, solvate, hydrate, co-crystal, clathrate, or polymorph thereof.

In some embodiments, the compound is avadomide, endomide, iberdomide, lenalidomide, mitindomide, pomalidomide, and thalidomide, or a derivative, analog, enantiomer or a mixture of enantiomers, or a pharmaceutically acceptable salt, solvate, hydrate, co-crystal, clathrate, or polymorph thereof.

In various embodiments, the first receptor and second receptor are the same. In various embodiments, the first receptor and second receptor are different.

In some embodiments, the ligand-binding domain is derived from a cytokine receptor. In some embodiments, the ligand-binding domain is derived from a Type 1 cytokine receptor (CR). In other embodiments, the ligand-binding domain is derived from erythropoietin receptor (EpoR) or leptin receptor (LR). In some embodiments, the transmembrane and cytoplasmic domains are derived from the murine leptin receptor.

In some embodiments, the bait is heterologous to the first receptor and/or second receptor fragment. In some embodiments, the cytoplasmic domain comprises a JAK binding site. In some embodiments, the cytoplasmic domain comprises glycoprotein 130 (gp130). In some embodiments, the receptor fragment comprises glycoprotein 130 (gp130). In some embodiments, the STAT is selected from STAT1 or STAT3.

In some embodiments, the mutations that reduce or eliminate STAT recruitment are to one or more tyrosine phosphorylation sites. In some embodiments, the transmembrane and cytoplasmic domains are derived from the murine leptin receptor and the mutations are at one or more of positions Y985, Y1077, and Y1138. In some embodiments, the transmembrane and cytoplasmic domains are derived from the murine leptin receptor and the mutations are Y985F, Y1077F, and Y1138F. In some embodiments, the transmembrane and cytoplasmic domains have functionally equivalent mutations to Y985F, Y1077F, and Y1138F of the murine leptin receptor.

In some embodiments, there is provided a deletion of a transmembrane domain, provided that JAK binding is retained.

The amino acid sequence of the murine leptin receptor is as follows:

(SEQ ID NO: 1)
MMCQKFYVVLLHWEFLYVIAALNLAYPISPWKFKLF
CGPPNTTDDSFLSPAGAPNNASALKGASEAIVEAK
FNSSGIYVPELSKTVFHCCFGNEQGQNCSALTDNT
EGKTLASVVKASVFRQLGVNWDIECWMKGDLTLFI
CHMEPLPKNPFKNYDSKVHLLYDLPEVIDDSPLPP
LKDSFQTVQCNCSLRGCECHVPVPRAKLNYALLMY
LEITSAGVSFQSPLMSLQPMLVVKPDPPLGLHMEV
TDDGNLKISWDSQTMAPFPLQYQVKYLENSTIVRE
AAEIVSATSLLVDSVLPGSSYEVQVRSKRLDGSGV
WSDWSSPQVFTTQDVVYFPPKILTSVGSNASFHCI
YKNENQIISSKQIVWWRNLAEKIPEIQYSIVSDRV
SKVTFSNLKATRPRGKFTYDAVYCCNEQACHHRYA
ELYVIDVNINISCETDGYLTKMTCRWSPSTIQSLV
GSTVQLRYHRRSLYCPDSPSIHPTSEPKNCVLQRD
GFYECVFQPIFLLSGYTMWIRINHSLGSLDSPPTC
VLPDSVVKPLPPSNVKAEITVNTGLLKVSWEKPVF
PENNLQFQIRYGLSGKEIQWKTHEVFDAKSKSASL
LVSDLCAVYVVQVRCRRLDGLGYWSNWSSPAYTLV
MDVKVPMRGPEFWRKMDGDVTKKERNVTLLWKPLT
KNDSLCSVRRYVVKHRTAHNGTWSEDVGNRTNLTF
LWTEPAHTVTVLAVNSLGASLVNFNLTFSWPMSKV
SAVESLSAYPLSSSCVILSWTLSPDDYSLLYLVIE
WKILNEDDGMKWLRIPSNVKKFYIHDNFIPIEKYQ
FSLYPVFMEGVGKPKIINGFTKDAIDKQQNDAGLY
VIVPIIISSCVLLLGTLLISHQRMKKLFWDDVPNP
KNCSWAQGLNFQKPETFEHLFTKHAESVIFGPLLL
EPEPISEEISVDTAWKNKDEMVPAAMVSLLLTTPD
PESSSICISDQCNSANFSGSQSTQVTCEDECQRQP
SVKYATLVSNDKLVETDEEQGFIHSPVSNCISSNH
SPLRQSFSSSSWETEAQTFFLLSDQQPTMISPQLS
FSGLDELLELEGSFPEENHREKSVCYLGVTSVNRR
ESGVLLTGEAGILCTFPAQCLFSDIRILQERCSHF
VENNLSLGTSGENFVPYMPQFQTCSTHSHKIMENK
MCDLTV.

In some embodiments, the domains are derived from the murine leptin receptor are amino acids 839-1162 of the murine leptin receptor sequence.

In some embodiments, the prey protein comprises a nuclear export sequence (NES). For example, in embodiments, the prey protein is a nuclear protein and the NES ensures that it is available in the cytosol (i.e. to contact the bait, if applicable). Thus, in embodiments, the NES signal helps keep the prey polypeptide in the cytoplasm even when a strong nuclear localization signal is present, thus facilitating interaction with the bait protein.

In some embodiments, the NES has 1˜4 hydrophobic residues. In some embodiments, the hydrophobic residues are leucines. In some embodiments, the NES has the sequence LxxxLxxLxL, where L is a hydrophobic residue and x is any other amino acid. In some embodiments, the NES has the sequence LxxxLxxLxL, where L is a leucine and x is any other amino acid.

In some embodiments, the NES comprises amino acids 37-46 of the heat-stable inhibitor of the cAMP-dependent protein kinase, which has been shown to override a strong nuclear localization signal (Wiley et al., (1999), J. Biol. Chem. 274:6381-6387, the entire contents of which are incorporated by reference).

In some embodiments, the interactions between the bait protein, the small molecule and the prey protein, or a combination thereof is monitored or detected in the presence of a proteasome inhibitor. In one embodiment, the method includes providing a proteasome inhibitor to the cell. In some embodiments, the proteasome inhibitor inhibits potential degradation of the prey protein in the event that it gets modified upon its interaction with the bait protein comprising an E3 ligase component. The proteasome inhibitor for use in the methods disclosed herein could be selected from carfilzomib (Kyprolis), bortezomib (Velcade), ixazomib (Ninlaro), and marizomib. In one embodiment, the proteasome inhibitor is bortezomib (Velcade).

In various embodiments, the present methods identify a novel molecular interaction. In various embodiments, the present methods identify a novel protein/protein interaction. In various embodiments, the present methods identify a novel protein/protein interaction which is mediated by the binding of a small molecule with the prey protein or bait protein.

In various embodiments, the present methods identify a molecular interaction without the need for using a hybrid ligand (or small molecule or compound) or a ligand where two small molecule entities are joined together by a linker. In embodiments, the small molecule is a single chemical entity. In embodiments, the small molecule does not have a linker.

In embodiments, the small molecule only directly interacts with one of the bait or prey protein. In embodiments, the small molecule directly interacts with the bait and/or prey protein but only in the presence of the bait or prey protein, e.g. the small molecule directly interacts with the prey protein but only in the presence of the bait protein, or the small molecule directly interacts with the bait protein but only in the presence of the prey protein, or the small molecule directly interacts with both of the bait or prey protein but only in the presence of the bait or prey protein.

In some embodiments, the present methods are applicable to the use of VHL as a E3 ligase substrate binding bait protein. VHL is, similarly to CRBN, the substrate binding subunit of an E3 ligase. Accordingly all embodiments relating to an E3 ligase as bait are equally applicable to VHL as bait.

In embodiments, the present methods are applicable to the use FKBP12 protein or a member of this family (e.g. FK506 binding proteins), instead of an E3 ligase, as bait (accordingly all embodiments relating to E3 ligase as bait are equally applicable to FKBP12 protein or a member of this family as bait).

FKBP12 is known to bind the immunosuppressant molecule tacrolimus (FK506). In embodiments, the small molecule is FK506 or a derivative, analog, enantiomer or a mixture of enantiomers, or a pharmaceutically acceptable salt, solvate, hydrate, co-crystal, clathrate, or polymorph thereof.

The invention is further described with the following non-limiting examples.

In embodiments, there is provided a method for detecting a molecular interaction, comprising: (a) providing a cell comprising a ligand-dependent chimeric receptor comprising: (i) an extracellular portion of a ligand-binding domain derived from a first receptor and (ii) transmembrane and cytoplasmic domains of a second receptor and a intracellular bait protein fused thereto, wherein the transmembrane and/or cytoplasmic domains of the second receptor comprise mutations that reduce or eliminate STAT (Signal Transducer and Activator of Transcription) recruitment; (b) expressing a prey protein that is fused to a receptor fragment in the cell, the receptor fragment comprising functional STAT recruitment sites; and (c) detecting a signal that is indicative of a molecular interaction, where the bait protein is an FK506 binding protein (FKBP).

In embodiments, the interaction between the prey protein and bait protein causes recruitment of the receptor fragment fused to the bait protein to the transmembrane chimeric receptor protein, which restores ligand-dependent transmembrane chimeric receptor signaling and activation of STAT molecules.

In embodiments, the cell comprises a STAT-responsive reporter gene.

In embodiments, the activated STAT molecules migrate to the nucleus and induce transcription of the STAT-responsive reporter gene, the reporter gene signal permitting detection of a molecular interaction.

In embodiments, the FK506 binding protein (FKBP) is selected from FKBP12, FKBP38 and FKBP52.

In embodiments, the method further comprises introducing a small molecule which binds to the prey protein and/or bait protein.

In embodiments, the molecular interaction is a protein/protein interaction which is mediated by the binding of the small molecule with the prey protein or bait protein.

In embodiments, the molecular interaction is two or more protein/protein interactions which are mediated by the binding of the small molecule with the prey protein or bait protein.

In embodiments, the protein/protein interaction which is mediated by the binding of the small molecule with the prey protein or bait protein is a direct binding between the prey protein or bait protein and the small molecule at a protein/protein interface.

In embodiments, the protein/protein interaction which is mediated by the binding of the small molecule with the prey protein or bait protein is mediated by an allosteric modification of the protein surface of the bait protein.

In embodiments, the small molecule induces exposure of a hydrophobic surface of the bait protein that allows for interaction with the prey protein.

In embodiments, the small molecule is a molecular glue.

In embodiments, the molecular interaction is a complex formation.

In embodiments, the molecular interaction is a small molecule/protein interaction.

In embodiments, the first receptor and second receptor are the same.

In embodiments, the first receptor and second receptor are different.

In embodiments, the first receptor and/or second receptor is a multimerizing receptor.

In embodiments, the ligand-binding domain is derived from a cytokine receptor.

In embodiments, the ligand-binding domain is derived from a Type 1 cytokine receptor (CR).

In embodiments, the ligand-binding domain is derived from erythropoietin receptor (EpoR) or leptin receptor (LR).

In embodiments, the transmembrane and cytoplasmic domains are derived from the murine leptin receptor (LR).

In embodiments, the bait is heterologous to the first receptor and/or second receptor fragment.

In embodiments, the cytoplasmic domain comprises a JAK binding site and/or the receptor fragment comprises gp130.

In embodiments, the STAT is selected from STAT1 or STAT3.

In embodiments, the mutations that reduce or eliminate STAT recruitment are to one or more tyrosine phosphorylation sites.

In embodiments, the transmembrane and cytoplasmic domains are derived from the murine leptin receptor (LR) and the mutations are at one or more of positions Y985, Y1077, and Y1138.

In embodiments, the transmembrane and cytoplasmic domains are derived from the murine leptin receptor (LR) and the mutations are Y985F, Y1077F, and Y1138F.

In embodiments, the transmembrane and cytoplasmic domains have functionally equivalent mutations to Y985F, Y1077F, and Y1138F of the murine leptin receptor (LR).

In embodiments, the prey protein comprises a nuclear export sequence (NES).

In embodiments, the NES has 1-4 hydrophobic residues.

In embodiments, the hydrophobic residues are leucines.

In embodiments, the NES has the sequence LxxxLxxLxL, where L is a hydrophobic residue and x is any other amino acid.

In embodiments, the NES has the sequence LxxxLxxLxL, where L is a leucine and x is any other amino acid.

In embodiments, the bait is contacted with a compound before interaction with the prey protein.

In embodiments, the compound is selected from FK506 (tacrolimus), rapamycin (sirolimus), and cyclosporin A (CsA) or a derivative or analog thereof or a compound that binds to the same FKBP bait binding site as the FK506 (tacrolimus), rapamycin (sirolimus), and cyclosporin A (CsA) or a derivative or analog thereof and in a competitive fashion.

In embodiments, the method identifies a novel protein/protein interaction which is mediated by the binding of the small molecule with the prey protein or bait protein.

### EXAMPLES

### Example 1: Evaluation of a MAPPIT-Derivative Assay Configuration for the Detection of Molecular Glue-Induced CRBN Substrate Interactions

In order to identify ligand-induced CRBN substrates, or neosubstrates, here we use a derivative of the MAPPIT assay, applying the procedure described in Lemmens, et al. “MAPPIT, a mammalian two-hybrid method for in-cell detection of protein-protein interactions,” Methods Mol Biol. 2015; 1278:447-55. The traditional MAPPIT assay has been used to monitor protein-protein interactions. A bait protein (protein A) is expressed as a fusion protein in which it is genetically fused to an engineered intracellular receptor domain of the leptin receptor, which is itself fused to the extracellular domain of the erythropoietin (Epo) receptor. Binding of Epo ligand to the Epo receptor component results in activation of receptor-associated intracellular JAK2. However, activated JAK2 cannot activate the leptin receptor to trigger STAT3 binding and its phosphorylation because its tyrosine residues, normally phosphorylated by activated JAK2, have been mutated. Reconstitution of a JAK2 phosphorylatable STAT3 docking site is instead created through interaction of a protein B with protein A, whereby protein B is fused to a cytoplasmic domain of the gp130 receptor (which now harbors appropriate tyrosine resides recognized by the activated JAK2 kinase). Thus, physical interaction of protein A with protein B reconstitutes an EPO triggered JAK2-STAT3 signaling pathway activation. Activation of STAT3 can be monitored by introduction of a STAT3-responsive reporter gene, including a luciferase-encoding gene or a gene encoding a fluorescent marker such as GFP or some other type of Fluorescent Protein (EGFP, etc.). In this manner, the MAPPIT assay provides a versatile assay to assess such recombinant protein-protein interactions in intact cells.

In this Example 1, we used a derivative of the MAPPIT assay that we developed specifically for use in determining CRBN-ligand induced protein interactions, i.e. using a specific CRBN bait protein and assaying for ligand-dependent induction of protein complex formation. The CRBN bait protein is expressed as a fusion with the MAPPIT chimeric membrane receptor and the interacting target protein is fused with the cytoplasmic gp130 receptor fragment (gp130-IKZF1(isoform 7), gp130-target X, gp130-GSPT1(domain 2+3) or gp130-GSPT2). We evaluated this MAPPIT-derivative assay for detection of a panel of known IMiDs inducing recruitment of these substrates to CRBN (thalidomide, THL; lenalidomide, LEN; pomalidomide, POM; CC-122; CC-220; CC-885).

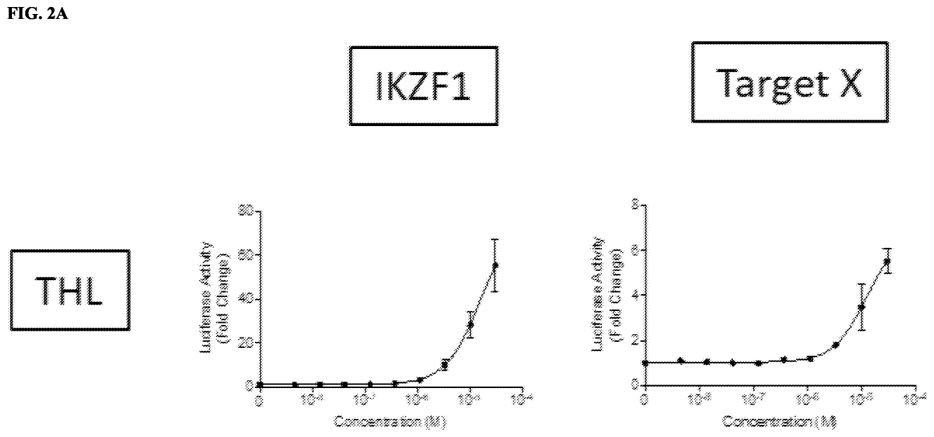

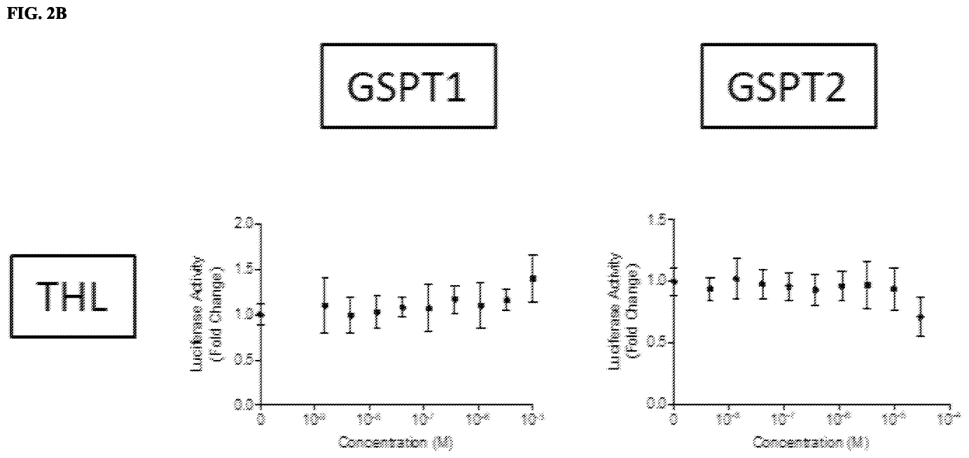

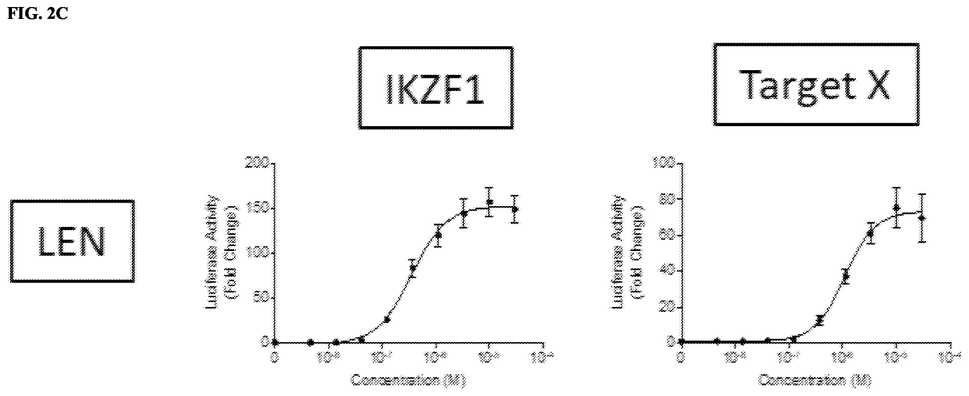

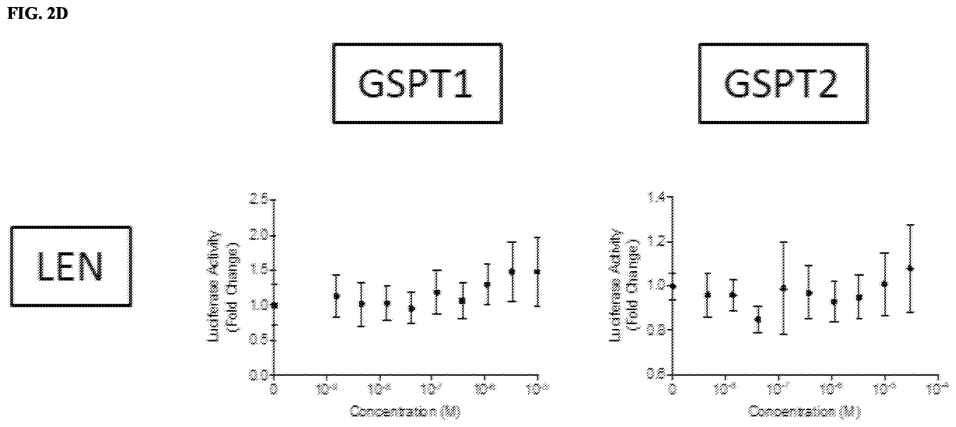

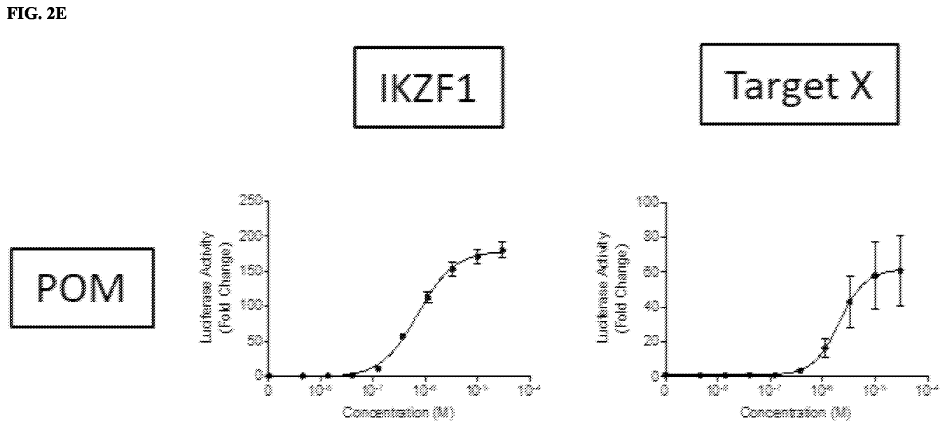

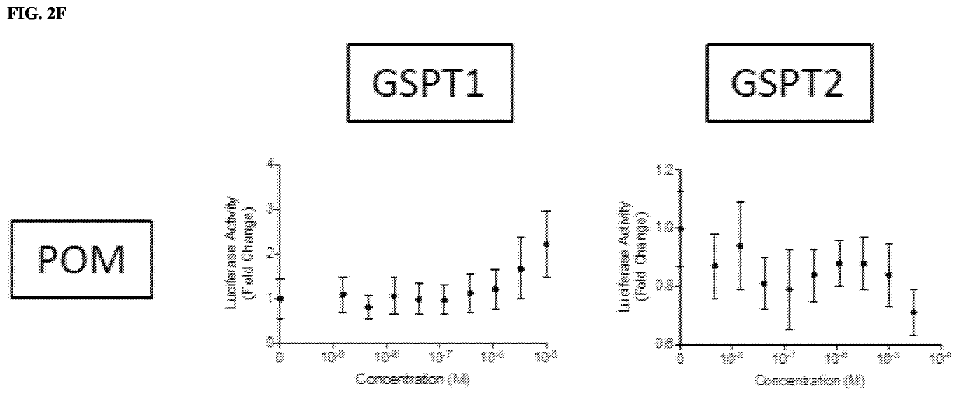

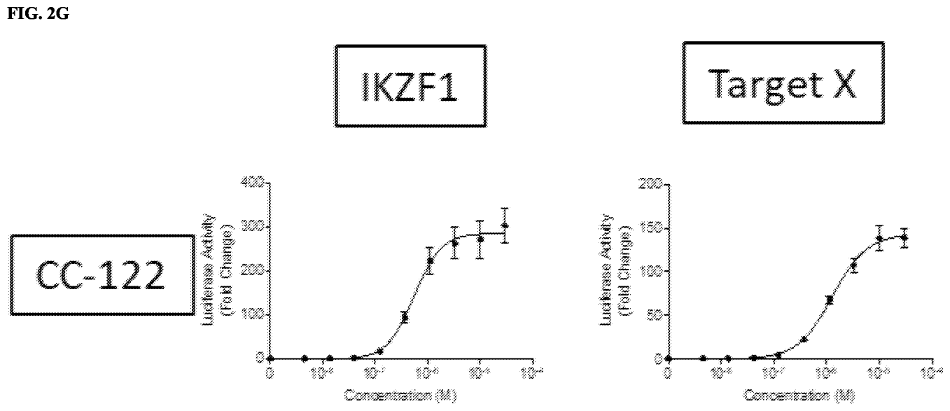

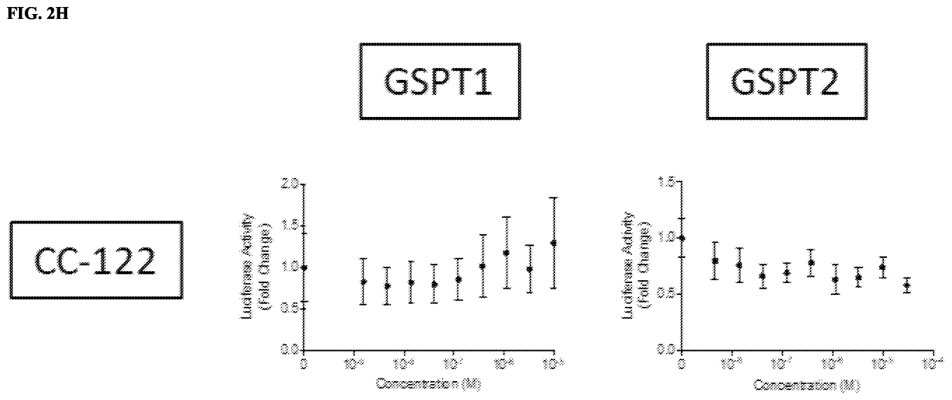

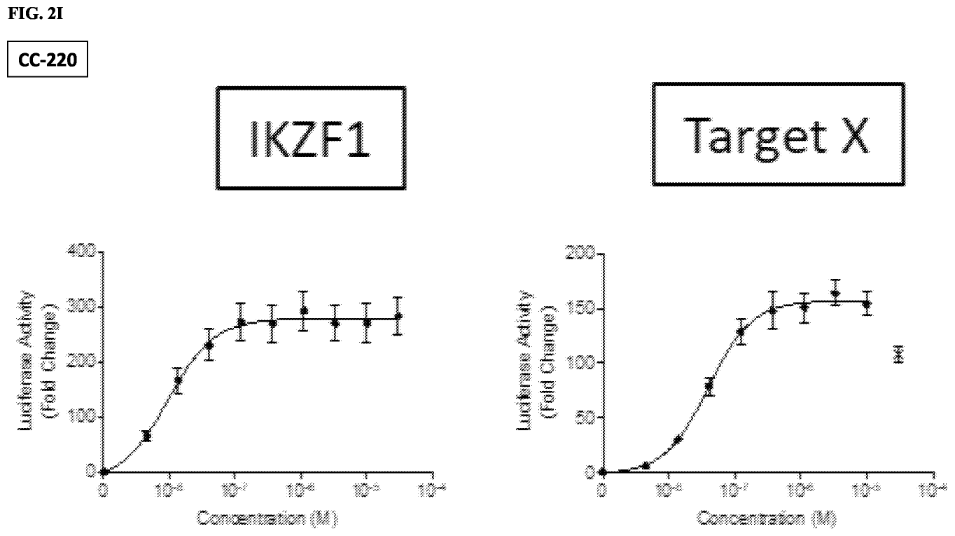

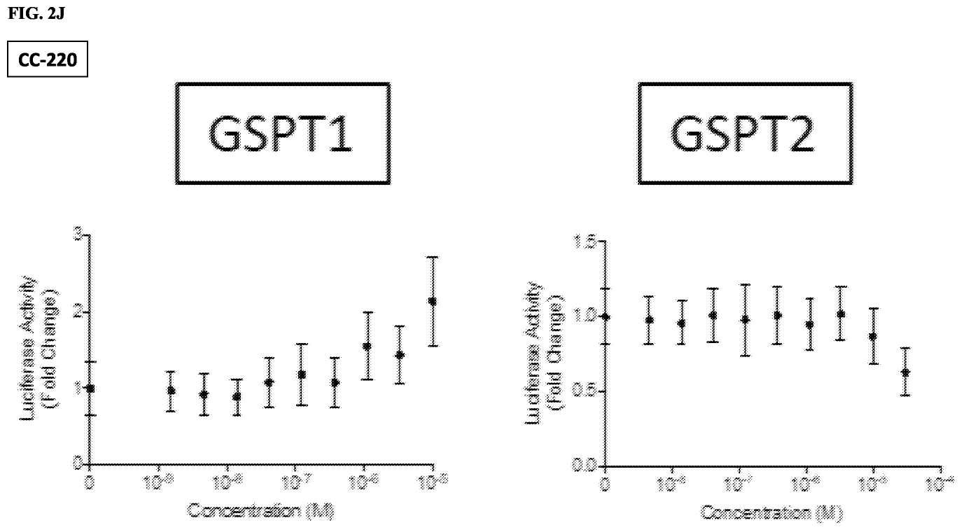

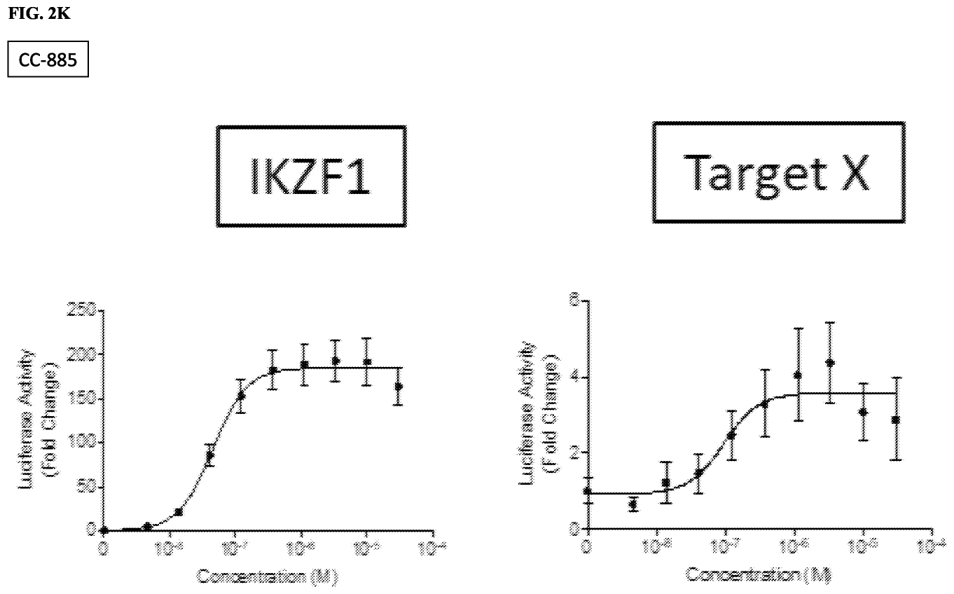

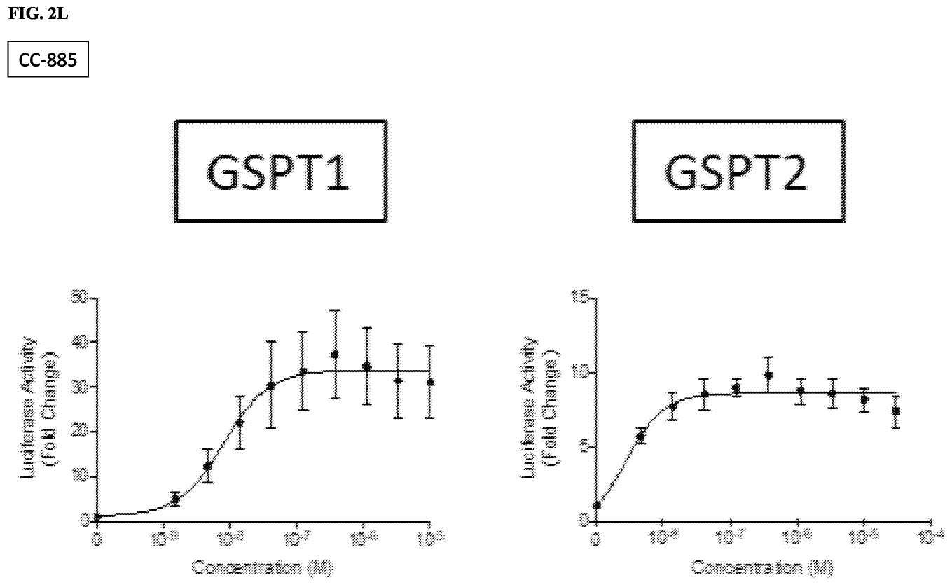

HEK293T cells were transfected with a plasmid encoding the CRBN chimeric receptor fusion (pSEL-CRBN), a plasmid encoding the MAPPIT gp130 fusion (gp130-IKZF1(isoform 7), gp130-target X, gp130-GSPT1(domain 2+3) or gp130-GSPT2) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Full size proteins were fused for each of the target proteins tested, except in the case of IKZF1 where isoform 7 was used and GSPT1, where an internal subdomain was used. The MAPPIT receptor fusion applied in this Example consists of the protein of interest (CRBN) genetically linked to the cytoplasmic domain of the leptin receptor, which itself is fused to the extracellular domain of the erythropoietin (EPO) receptor. The extracellular EPO receptor domain can be used interchangeably with the extracellular leptin receptor domain (as used in Example 2) to promote receptor/receptor-associated JAK2 activation (with EPO or Leptin, respectively). Cells were treated with erythropoietin (EPO) without or with the indicated dose of test compound at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. Curves were fit using 4-parameter nonlinear regression in GRAPHPAD PRISM software. The data shown in FIGS. 2A-L indicate that the MAPPIT recruitment assay is able to reproduce known interactions, MED specificity (e.g. GSPT1 and GSPT2 are only recruited through CC-885) and potency trends.

### Example 2: Comparison of Alternative CRBN MAPPIT-Derivative Receptor Constructs for Detection of IMiD-Induced Substrate Recruitment

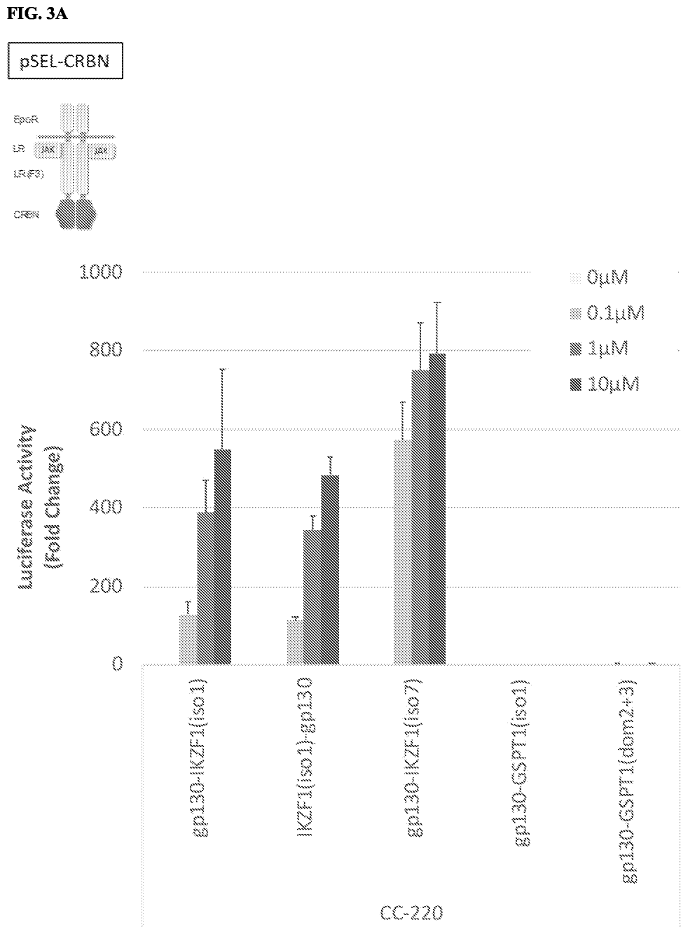

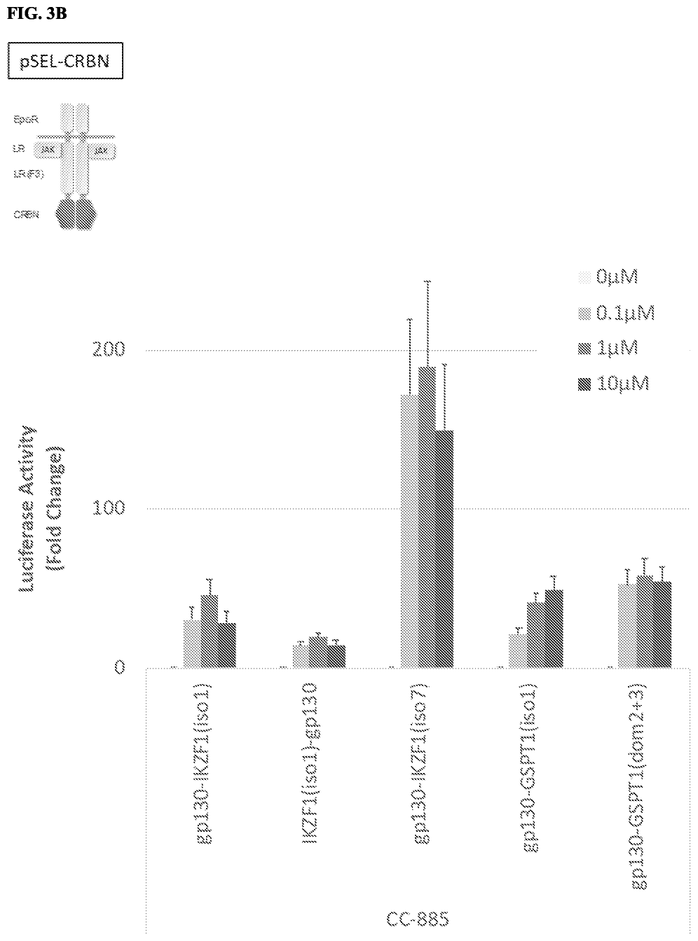

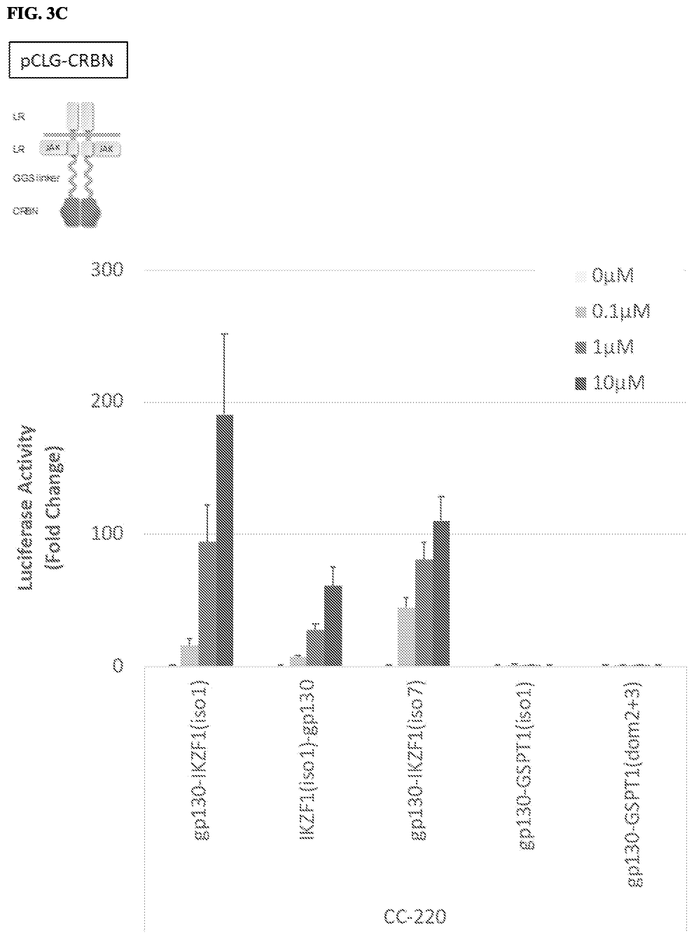

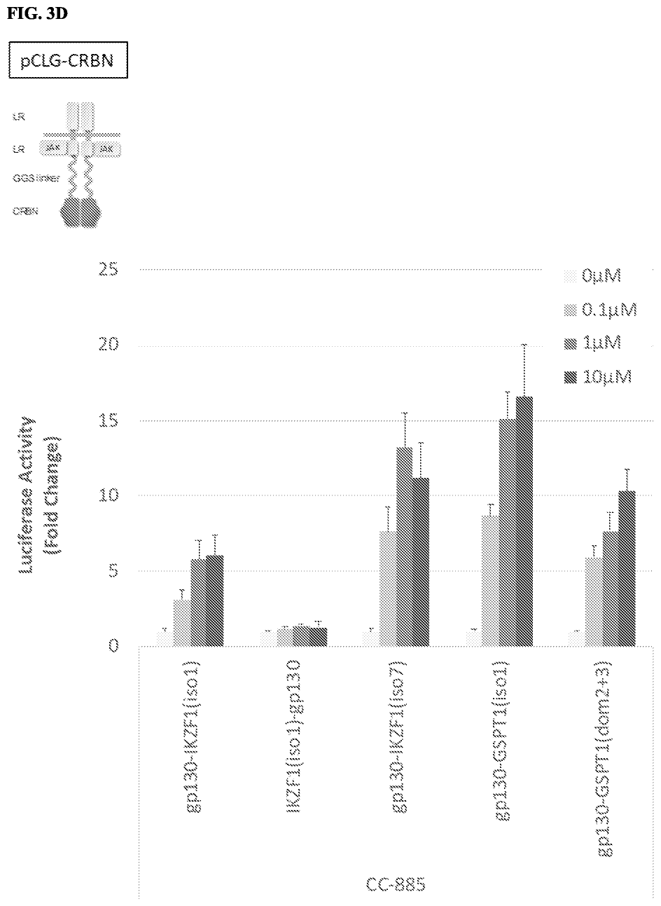

In this Example 2, the assay setup applied in Example 1 was tested side by side with a similar MAPPIT-derivative assay configuration where an alternative CRBN chimeric receptor fusion construct was used. As referred to already in Example 1, alternative receptor fusions are available where the EPO extracellular domain was exchanged for that of the leptin receptor, resulting in the assay system being activated by leptin instead of EPO. In the current example, HEK293T cells were transfected with a plasmid encoding CRBN tethered to a MAPPIT receptor fusion containing either the EPO receptor extracellular domain (pSEL-CRBN; as in Example 1) or the leptin receptor extracellular domain (pCLG-CRBN). As indicated in the cartoons in FIGS. 3A-D, apart from the extracellular domain, these constructs also differ in the intracellular configuration of the chimeric receptor. In the case of the pSEL-CRBN construct, the fusion contains the entire engineered leptin receptor intracellular domain, whereas in the pCLG-CRBN construct, a short portion of the leptin receptor is used harboring the JAK2 recruitment site and an additional Gly-Gly-Ser hinge is placed between this domain and the CRBN bait protein that is fused to it. In addition, the HEK293T cells were co-transfected with a plasmid encoding the substrate of interest fused to the partial gp130 domain (IKZF1 isoform 1, IKZF1 isoform 7, GSPT1 isoform 1 or GSPT1 domain 2+3) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). In the case of the IKZF1(isoform1) gp130 fusion, two different constructs were applied, with gp130 fused either to the N- or C-terminus of IKZF1. Cells were treated with EPO (in the case of pSEL-CRBN based assays) or leptin (for pCLG-CRBN based assays) without or with the indicated dose of test compound (CC-220 or CC-885) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO or leptin+test compound treated cells versus EPO or leptin only treated cells. Error bars represent standard deviations. The data presented in FIGS. 3A-D indicate that both alternative MAPPIT receptor fusions enable the detection of molecular glue dependent neosubstrate interactions with CRBN.

### Example 3: Detection of CRBN Compound-Induced Substrate Interactions Using a DDB1 MAPPIT-Derivative Receptor Fusion Construct

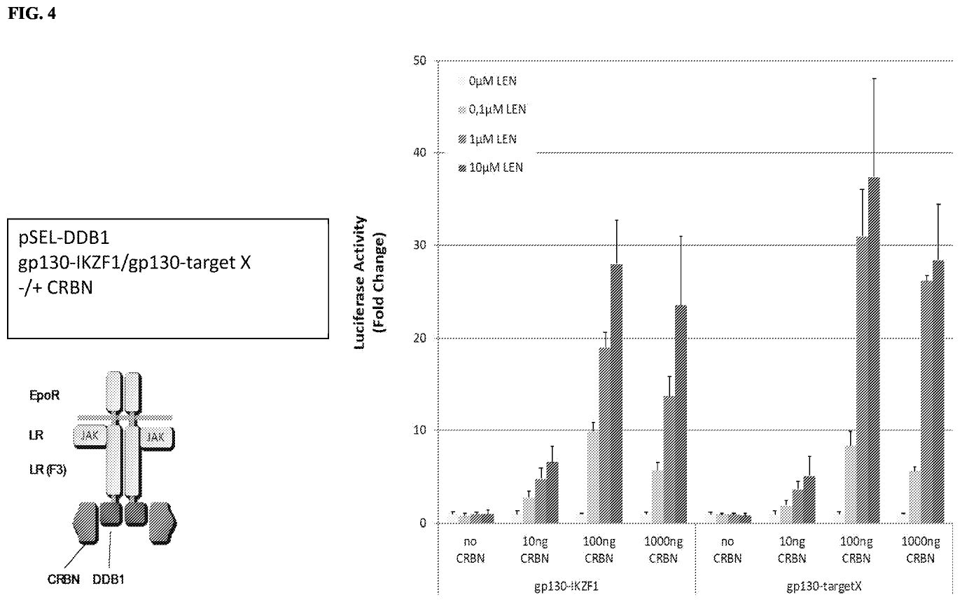

Since it may be advantageous to test compound-induced CRBN-substrate interactions using an unfused version of the CRBN bait, we developed a MAPPIT-derivative assay where DDB1 is fused to the MAPPIT chimeric receptor construct rather than CRBN. DDB1 is an adaptor protein that connects CRBN to the core E3 ubiquitin ligase complex scaffold subunit CUL4A or CUL4B (Cullin4A or Cullin 4B). HEK293T cells were transfected with a plasmid encoding DDB1 tethered to a MAPPIT receptor fusion containing the EPO receptor extracellular domain (pSEL-DDB1), a plasmid encoding a CRBN substrate protein (IKZF1 isoform 7 or the undisclosed target protein X also used in Example 1) fused to the partial gp130 domain and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). In addition, cells were also co-transfected with different amounts of an unfused CRBN expression construct. Cells were treated with EPO without or with the indicated dose of lenalidomide (LEN) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. As shown in FIG. 4, a robust lenalidomide-dependent MAPPIT signal is obtained for both IKZF1 and target X interactions, but only in the presence of co-expressed unfused CRBN, indicating that the signal is mediated by binding of the substrate gp130 fusion proteins to CRBN.

### Example 4: Enhanced Detection of CRBN Compound-Induced Substrate Interactions Upon Co-Expression of DDB1

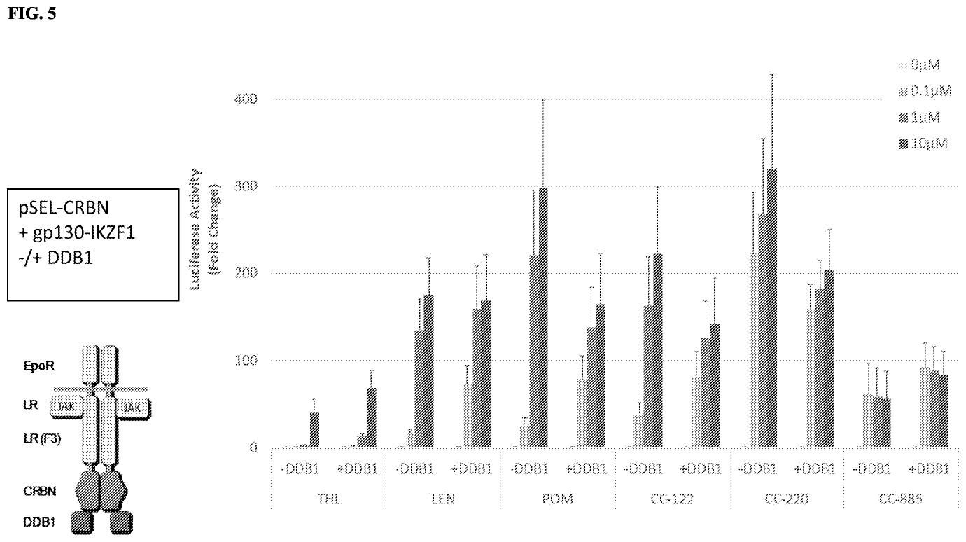

As the DDB1 adaptor protein is an essential component of the CRBN E3 ligase complex, linking CRBN to the CUL4A or CUL4B complex scaffold protein, endogenous DDB1 levels might be limiting in cells expressing the MAPPIT-derivative fusion protein components of a CRBN-substrate recruitment assay. In this Example 4, the effect of DDB1 co-expression was evaluated for the IMiD-induced interaction between CRBN and IKZF1. HEK293T cells were transfected with a plasmid encoding a CRBN MAPPIT receptor fusion containing the EPO receptor extracellular domain (pSEL-DDB1), a plasmid encoding a gp130 fusion of IKZF1 (isoform 7) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). In addition, in some of the conditions tested, cells were additionally co-transfected with an unfused DDB1 expression plasmid. Cells were treated with EPO without or with the indicated IMiD dose (thalidomide, THL; lenalidomide, LEN; pomalidomide, POM; CC-122; CC-220; CC-885) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. The data in FIG. 5 show that in the samples where DDB1 was co-expressed, signals were increased at the lower compound concentrations tested compared to the maximal signal for that compound, suggesting that co-expression of DDB1 improved assay sensitivity.

### Example 5: Detection of CRBN Compound-Induced Substrate Interactions Using a DDB1-CRBN MAPPIT Chimeric Receptor Fusion Construct

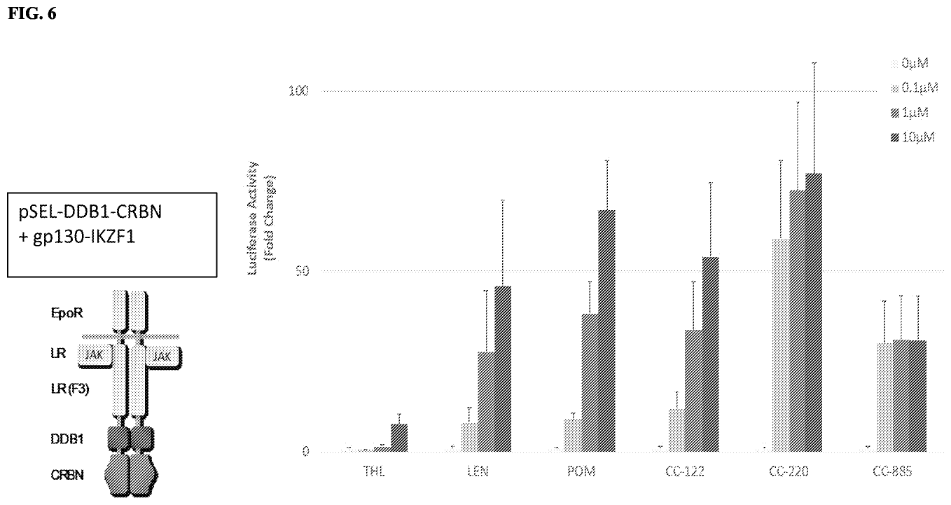

As discussed in Examples 3 and 4, DDB1 is a key component of the CRBN E3 ligase complex, essential for CRBN-mediated substrate recruitment and ubiquitination. In addition to the assay configurations applied in Examples 3 and 4, another MAPPIT-derivative assay setup uses a MAPPIT receptor construct containing a genetic fusion of DDB1 and CRBN. Such a fusion was generated with a MAPPIT chimeric receptor containing the EPO receptor extracellular domain (pSEL-DDB1-CRBN) and applied in this Example 5. This construct was transfected into HEK293T cells along with a plasmid encoding a gp130 fusion of IKZF1 (isoform 7) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO without or with the indicated MED dose (thalidomide, THL; lenalidomide, LEN; pomalidomide, POM; CC-122; CC-220; CC-885) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. The data in FIG. 6 indicate that a genetic DDB1-CRBN fusion can be applied in the MAPPIT-derivative assay to detect CRBN IMiD-induced substrate recruitment.

### Example 6: Evaluation of PROTAC-Induced Binding of Substrates to CRBN

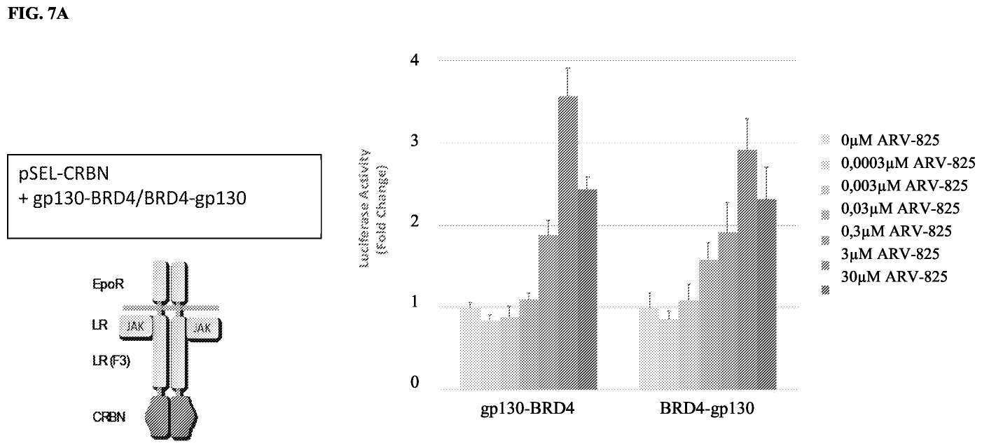

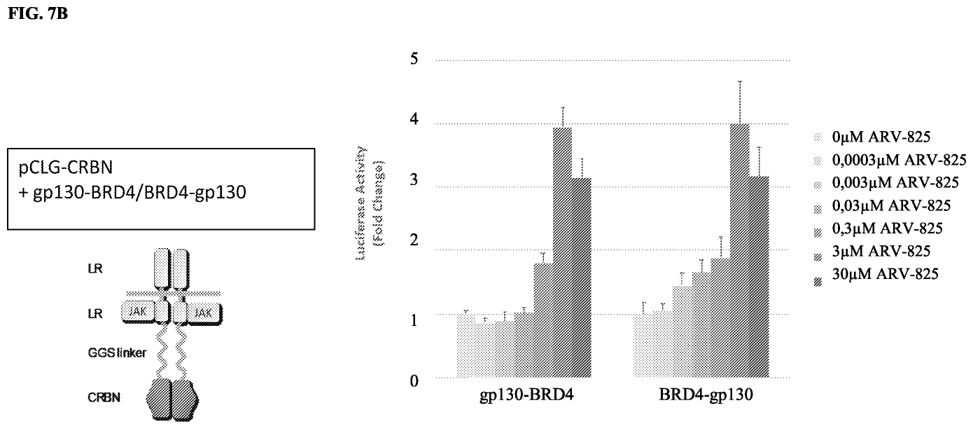

In this Example 6 we evaluated CRBN substrate recruitment induced by a Protac, which is a hybrid ligand constituted of a CRBN-binding ligand that is chemically tethered to a substrate binding ligand. The compound tested here is ARV-825, which is a chemical fusion of a CRBN binding ligand and a BRD4 binding compound. The two alternative MAPPIT-derivative CRBN bait receptor encoding plasmids applied in Example 2, encoding a fusion construct with either the EPO receptor extracellular domain (pSEL-CRBN) or the leptin receptor extracellular domain (pCLG-CRBN) were co-transfected in HEK293T cells together with a gp130 fusion of BRD4 (isoform 3 and the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO (in the case of the samples transfected with pSEL-CRBN) or leptin (for pCLG-CRBN) without or with the indicated dose of ARV-825 at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO or leptin+test compound treated cells versus EPO or leptin only treated cells. Error bars represent standard deviations. The data shown in FIGS. 7A-B show a clear dose-dependent signal increase in each of the assay configurations tested, indicating that the MAPPIT-derivative CRBN substrate recruitment assay is able to detect interactions induced by PROTAC-type molecules.

### Example 7: Detection of Compound-Induced FKBP1A (FKBP12) Substrate Interactions

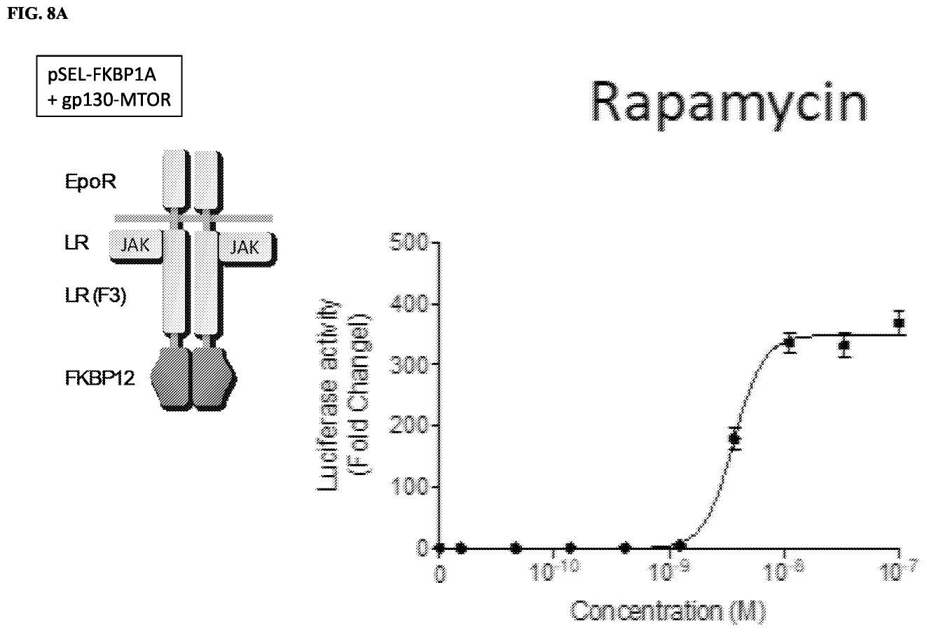

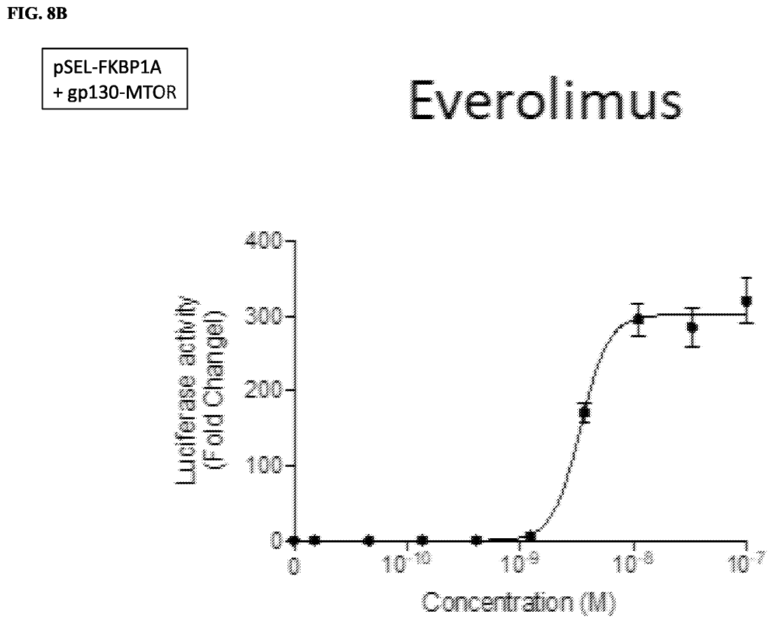

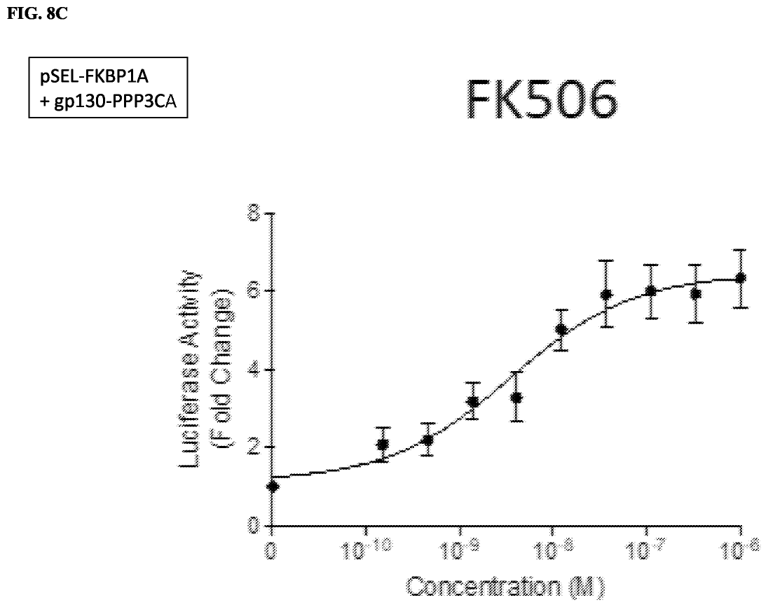

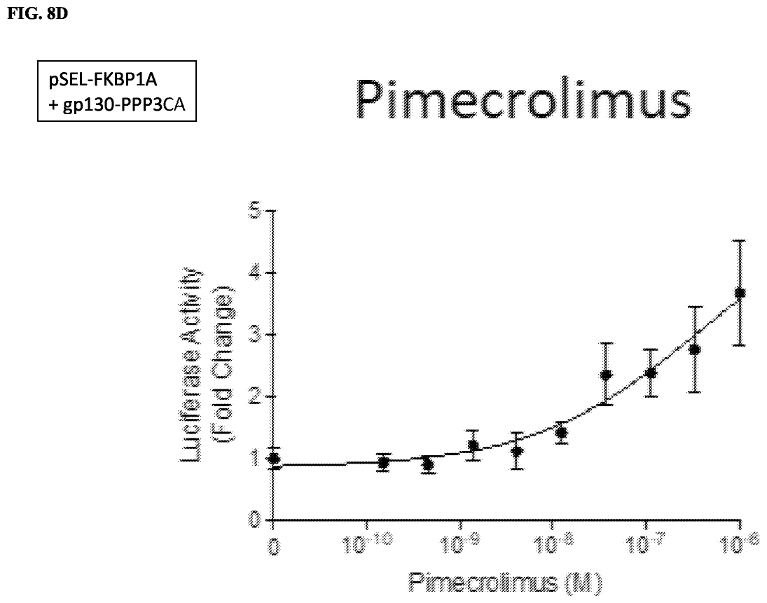

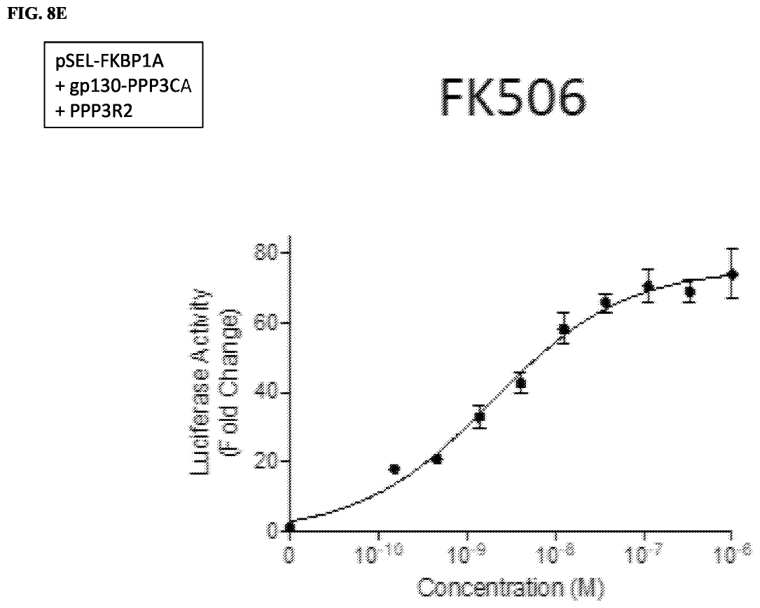

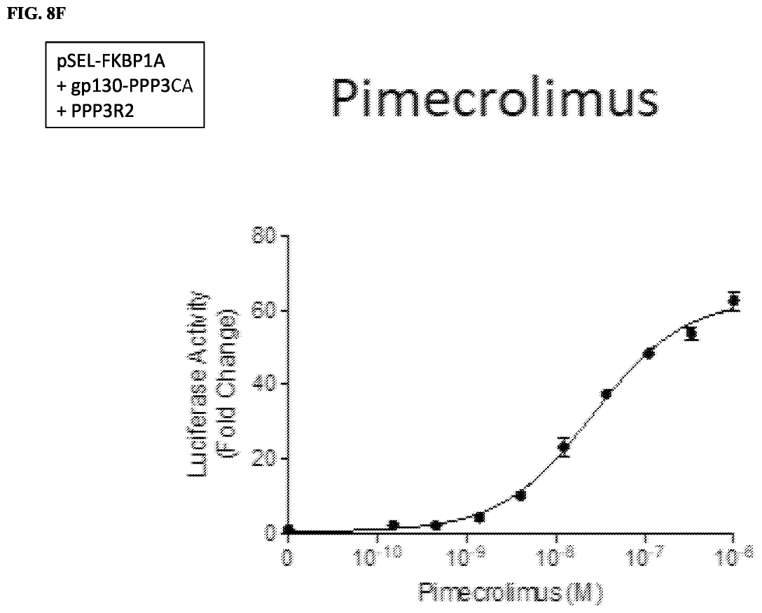

In this Example, a MAPPIT-derivative assay was applied for the detection of compound-dependent interactions of FKBP1A (FKBP12) with MTOR and calcineurin subunits. The experimental setup was according to the procedure described in Example 1, using the following plasmid constructs encoding the MAPPIT-derivative receptor and gp130 fusions: FKBP1A bait was fused to a MAPPIT chimeric receptor construct containing the extracellular EPO receptor domain (pSEL-FKBP1A) and the target proteins were fused to the partial gp130 domain (MTOR FRB domain or PPP3CA). For the calcineurin interaction, one additional assay setup was used where in addition to the MAPPIT receptor and gp130 fusions, an unfused PPP3R2-expressing plasmid was co-expressed, encoding a calcineurin regulatory subunit as it has been reported that this regulatory subunit contributes to the FK506 macrolide-induced FKBP1A-calcineurin interaction. HEK293T cells were transfected with the indicated receptor- and gp130-encoding plasmids and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO without or with the indicated dose of test compound (rapamycin or everolimus for MTOR recruitment; FK506 or pimecrolimus for calcineurin binding) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO only treated cells. Error bars represent standard deviations. Curves were fit using 4-parameter nonlinear regression in GRAPHPAD PRISM software. The results shown in FIGS. 8A-F indicate that the MAPPIT-derivative assay enables monitoring the compound-induced FKBP1A target binding. In the case of calcineurin recruitment, signal strength is significantly improved upon co-expression of the PPP3R2 regulatory subunit.

### Example 8: Detection of Compound-Induced VHL Substrate Interactions

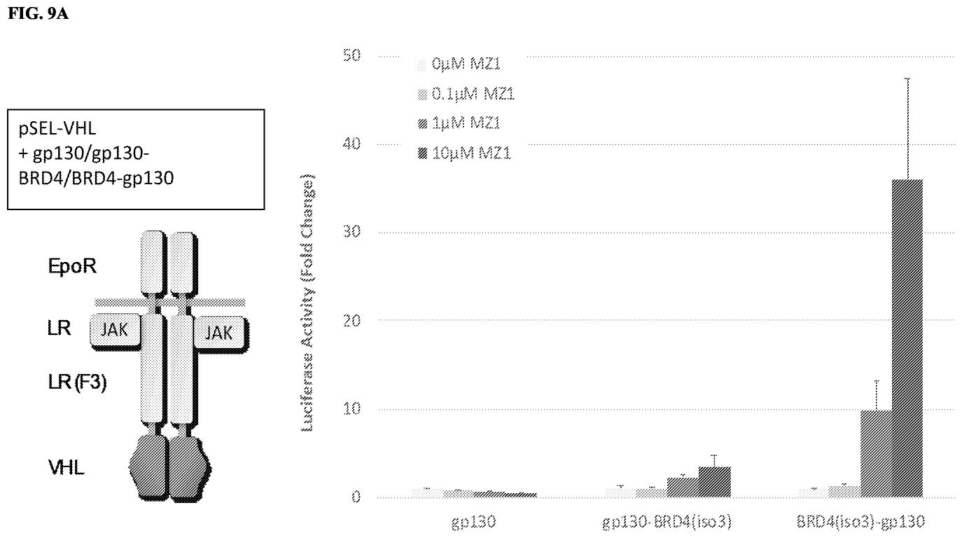

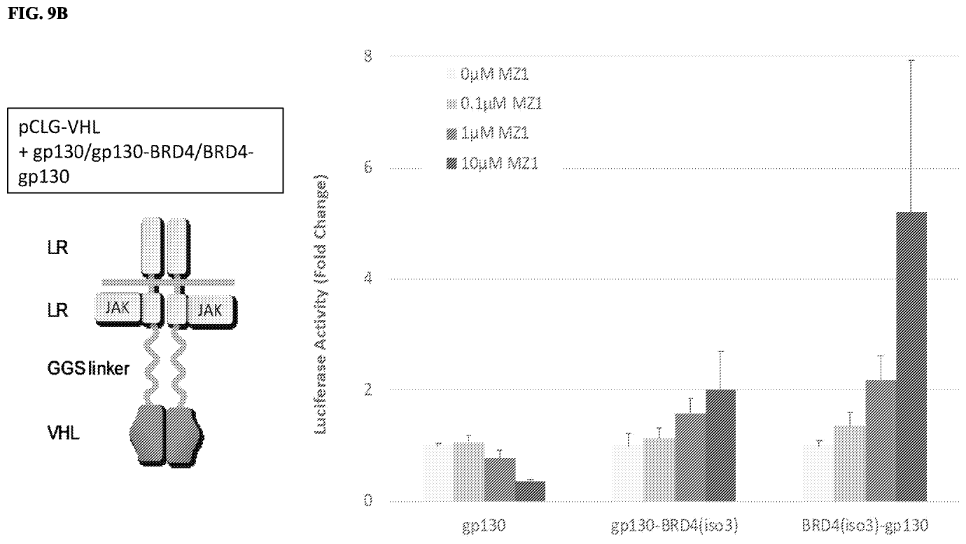

Similar to the Example 6, we applied a MAPPIT-derivative assay for the detection of Protac-dependent interactions of VHL with BRD4. Two alternative MAPPIT-derivative VHL bait receptor encoding plasmids, encoding a fusion construct with either the EPO receptor extracellular domain (pSEL-VHL) or the leptin receptor extracellular domain (pCLG-VHL) were co-transfected in HEK293T cells together with a gp130 fusion of BRD4 (isoform 3) and the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO (in the case of the samples transfected with pSEL-CRBN) or leptin (for pCLG-CRBN) without or with the indicated dose of MZ1 (a chemical fusion between a VHL and a BRD4 ligand) at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO or leptin+test compound treated cells versus EPO or leptin only treated cells. Error bars represent standard deviations. The graphs in FIGS. 9A-B show a clear dose-dependent signal increase in each of the assay configurations tested.

### Example 9: Compound Library Screening for the Identification of Novel Molecular Glues Inducing IKZF1 Recruitment to CRBN

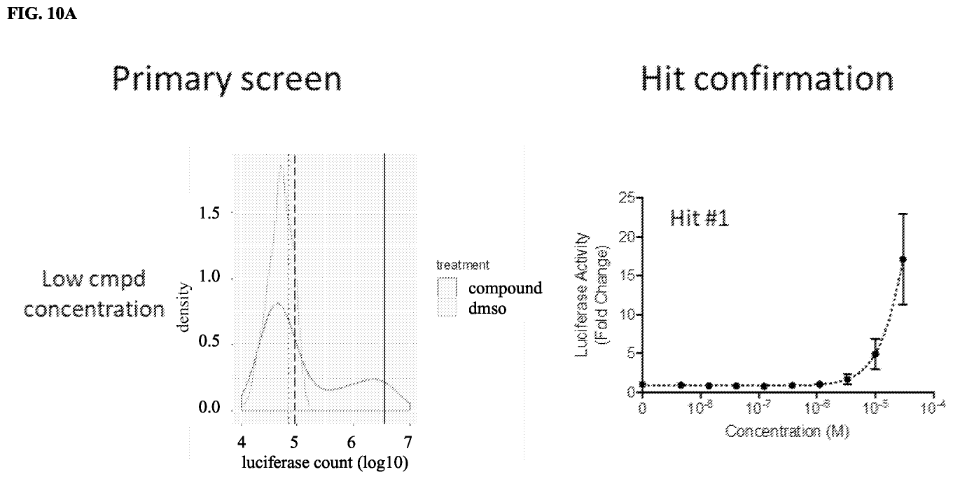

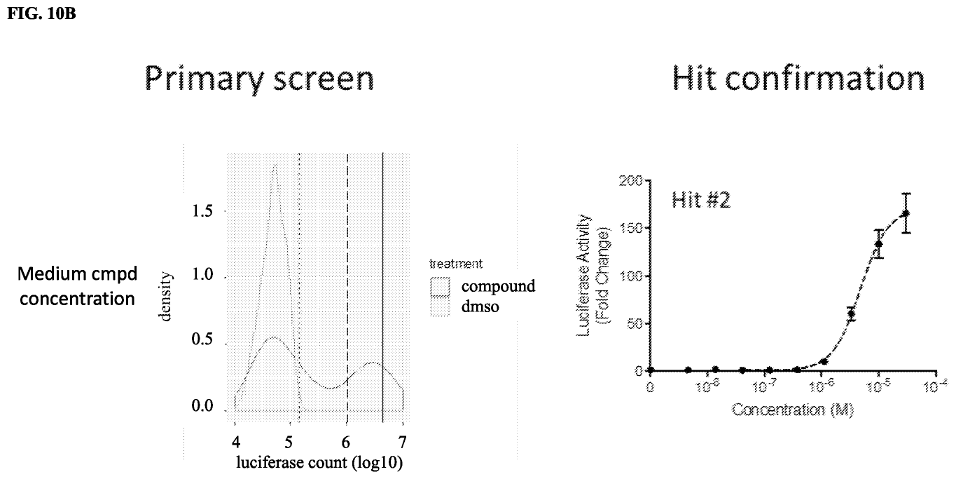

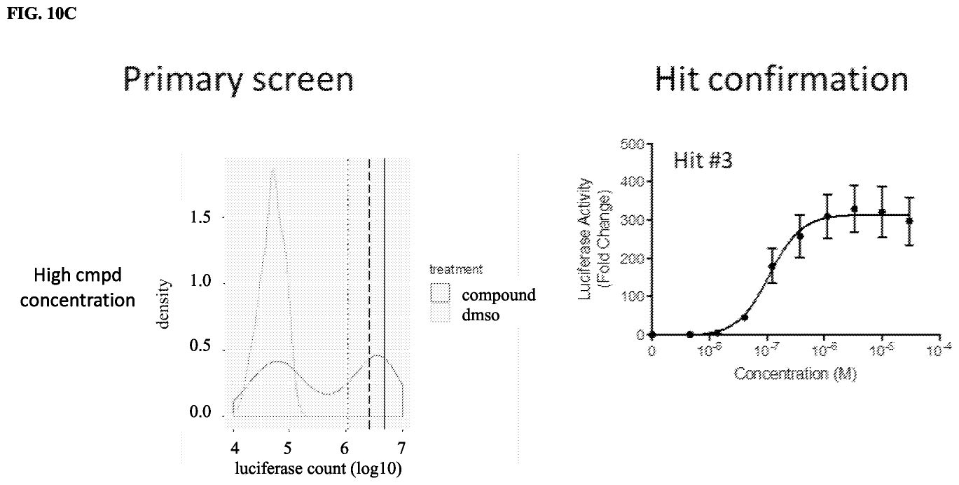

In this example, a compound collection consisting of 96 IMiDs and MED-like molecular glues was screened in microtiter plate format to identify compounds that induce recruitment of IKZF1 to CRBN, using the MAPPIT-derivative IKZF1-CRBN recruitment assay applied in Example 1. HEK293T cells were co-transfected with a plasmid encoding a fusion construct of the CRBN bait protein tethered to the chimeric MAPPIT-derivative receptor containing the EPO receptor extracellular domain (pSEL-CRBN) and a gp130-IKZF1 (isoform 7) fusion construct, together with the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO and compound (or DMSO as negative control) at 24 hours after transfection. Three concentrations (indicated as ‘low’, ‘medium’ and ‘high’ in FIGS. 10A-C) were applied for each compound: either 0.8, 4 and 20 μM or 0.2, 1 and 5 μM, depending on the previously assessed cellular toxicity level of the compound, and each compound concentration was tested in duplicate. Luciferase activity was measured 24 hours after compound treatment using the Luciferase Assay System kit (PROMEGA, Madison, Wis.) with an Ensight plate reader (PERKIN ELMER LIFE SCIENCES, Waltham, Mass.). The graphs shown in FIGS. 10A-C (left panel) depict the frequency distributions of the average raw luciferase signal for both the compound-treated samples and the DMSO-treated controls, and each graph corresponds with the data for one of the three tested compound concentrations (low, medium and high). The right-shifted portion of the bimodal distribution corresponding to the compound-treated samples represents those compounds with a signal above background and therefore inducing IKZF1 recruitment to CRBN. For three such compounds exhibiting a reporter signal above background for one or more of the three tested concentrations, the luciferase signal is indicated by line marks (dotted, dashed or solid) and the corresponding dose-response curves are shown (right panel). These dose-response curves were generated using the same assay setup and protocol used for the primary screen, but now testing a 9-point dose-range of the indicated concentrations. Here, data points depict fold induction of the average luciferase activity of triplicate samples from EPO+test compound treated cells versus EPO only treated cells. Error bars represent standard deviations and curves were fit using 4-parameter nonlinear regression in GRAPHPAD PRISM software. In summary, this example shows that the MAPPIT-derivative assay presented here can be applied to screen compound collections to identify known and novel molecule glues inducing substrate recruitment to CRBN. FIGS. 10A-C exemplifies compound screening for glue inducing IKZF1 recruitment to CRBN specifically, but the approach can be applied to screen any other potential substrate.

### Example 10: Identification of Novel Molecular Glue-Induced CRBN Substrates Using a MAPPIT-Derivative ORF cDNA Library Screening Approach

In order to identify ligand-induced CRBN substrates, or neosubstrates, a MAPPIT cell microarray screen was performed using the procedure described in Lievens, et al. “Proteome-scale binary interactomics in human cells.” Molecular & Cellular Proteomics 15.12 (2016): 3624-3639. In brief, HEK293T cells were transfected with the same CRBN bait expression plasmid (pSEL-CRBN) encoding a fusion construct of the CRBN bait protein tethered to the chimeric MAPPIT-derivative receptor containing the EPO receptor extracellular domain that was used in the previous Examples 1 and 9. These transfected cells were then added to microarray screening plates containing a prey gp130 fusion expression plasmid collection covering over 15,000 ORFs. Each spot in the microarray contained a different gp130-ORF fusion expression plasmid, as well as a STAT3-responsive fluorescence protein-encoding reporter plasmid. CRBN bait transfected cells landing and attaching on these spots therefore become transfected as well with the gp130-ORF prey plasmid and the reporter plasmid, resulting in a different CRBN-ORF combination being tested in the cells on every different microarray spot. Twenty-four hours after transfection cells were differentially stimulated with erythropoietin with and without the CRBN ligand CC-220 (10 μM), and reporter signal (GFP-like fluorescence reporter) was read out 48 hours later. Fluorescence intensity data was analyzed as reported previously, yielding a volcano plot where q-values calculated based on the integrated fluorescence intensity of each microarray cell cluster (Y-axis) are displayed against the ratio of the median value of the fluorescent particle count of the corresponding cell clusters (X-axis), as shown in FIGS. 11A-B. Four ORF cDNAs exhibiting a strong signal (indicated by arrows on the dot plot in FIGS. 11A-B) were selected for dose-response confirmation using the same assay setup and protocol as applied previously in Examples 1 and 9: the CRBN receptor fusion plasmid (pSEL-CRBN) was co-transfected in HEK293T cells together with the corresponding gp130-ORF plasmid and the luciferase reporter plasmid, 24 h after transfection the cells were treated with EPO without and with the indicated concentration of CC-220, and another 24 h later luciferase activity was determined. The dose-response curves represent the fold induction of the average luciferase activity of triplicate samples from EPO+test compound treated cells versus EPO only treated cells. Error bars represent standard deviations and curves were fit using 4-parameter nonlinear regression in GRAPHPAD PRISM software. As illustrated here for the case of CC-220, this example shows that the MAPPIT-derivative assay presented here can be applied to screen ORF cDNA collections to identify known and novel molecular glue-induced CRBN substrates.

### Example 11: Hybrid Ligand Library Screening for the Identification of Novel Ligands of a Protein of Interest

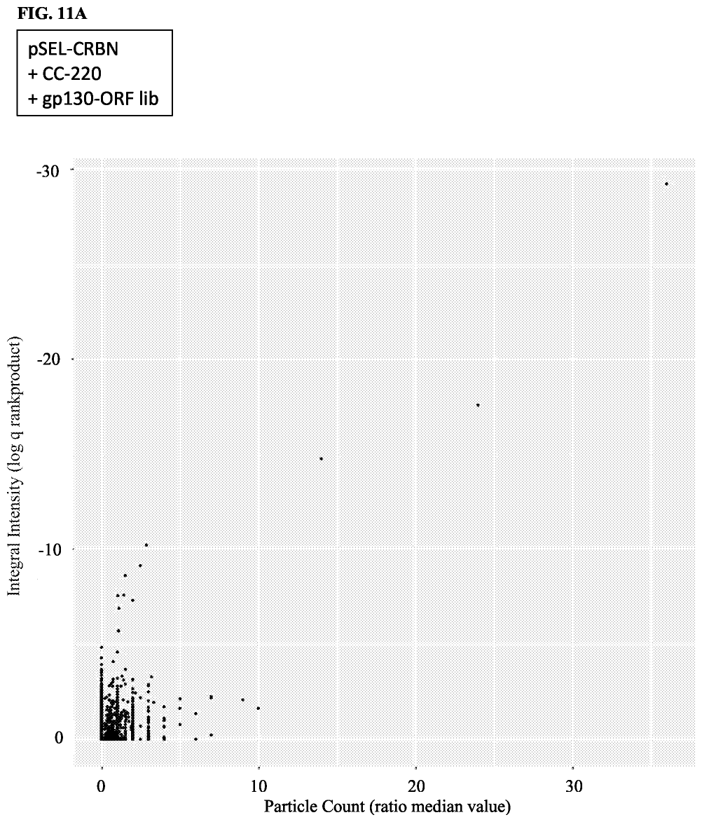

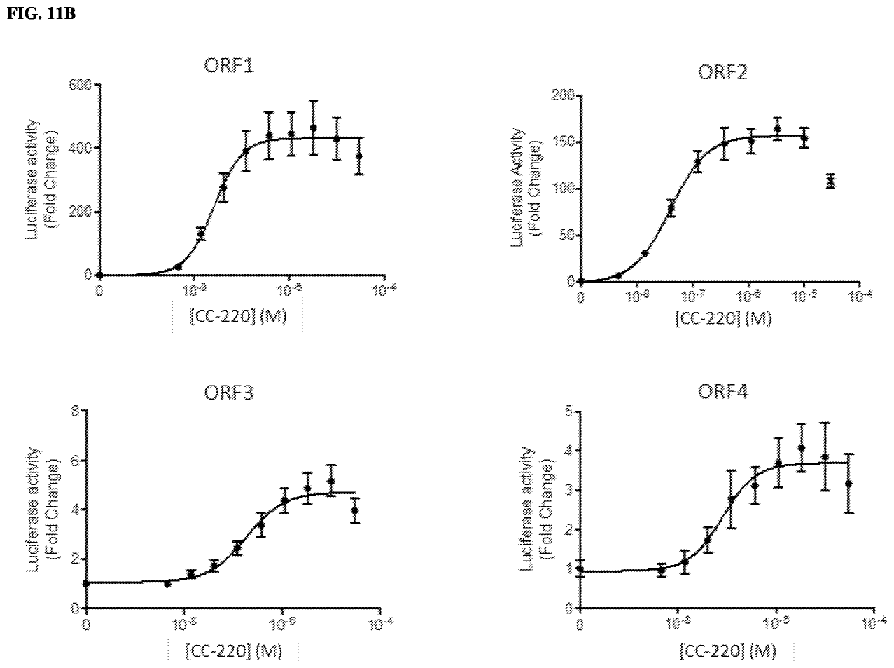

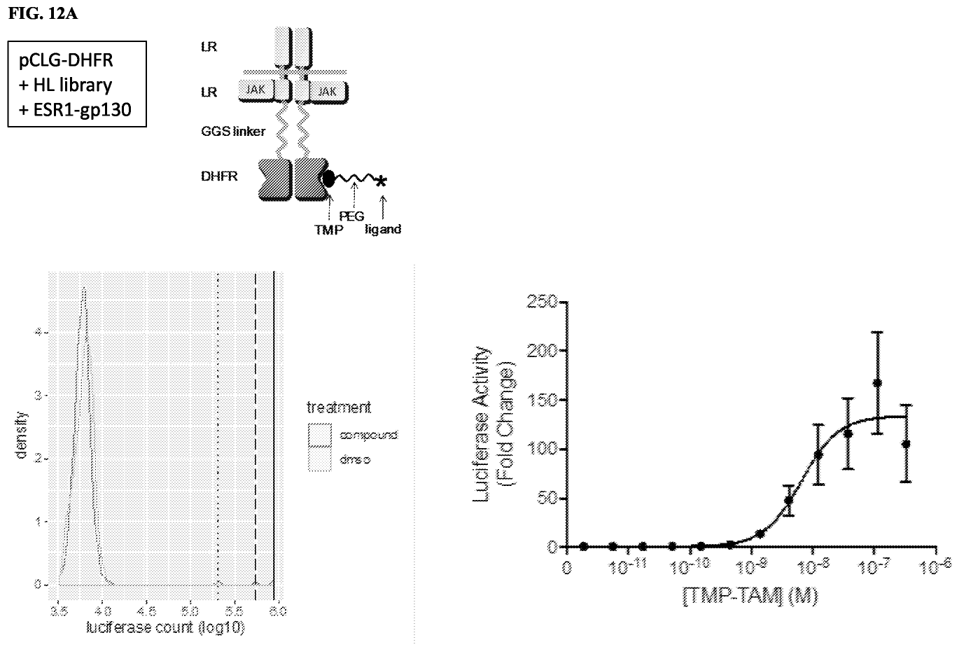

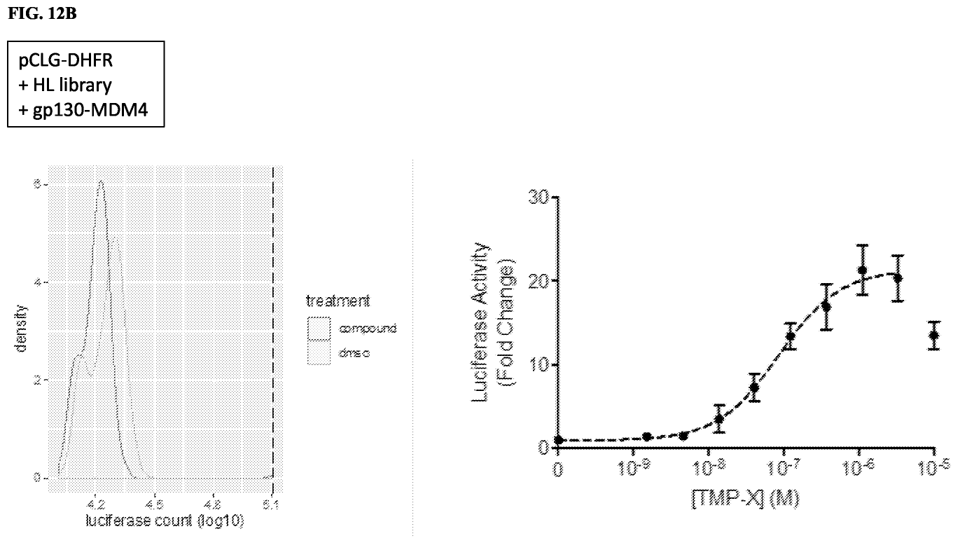

Similar to the approach described in Example 9, here a MAPPIT-derivative assay is used to screen a compound library in order to identify novel protein ligands. Here, in particular, a collection of trimethoprim (TMP)-ligand hybrid molecules was screened for binding to a protein of interest. Due to its tight binding with DHFR (dihydrofolate reductase), TMP can be used to anchor ligands as part of a TMP hybrid ligand fusion molecule to a MAPPIT-derivative DHFR receptor fusion and as such display the ligand as a bait (see cartoon in FIGS. 12A-B). In this assay setup, this DHFR receptor fusion is combined with the TMP hybrid ligand and a gp130-ORF fusion construct into a ternary complex resulting in a reporter signal. In this example, a hybrid ligand library is screened for compounds binding to the estrogen receptor (ESR1), a nuclear receptor and transcription factor that has been implicated in breast cancer and to MDM4 (Mouse Double Minute 4), an important cancer target involved in regulation of the p53 tumor suppressor. The compound collection screened here consisted of a 320 member hybrid ligand diversity set, spiked with TMP-TAM (tamoxifen), tamoxifen being a known ligand of ESR1. HEK293T cells were co-transfected with a plasmid encoding a fusion construct of the (E. coli) DHFR anchor protein tethered to the chimeric MAPPIT-derivative receptor containing the leptin receptor extracellular domain (pCLG-DHFR; see cartoon in FIGS. 12A-B) and either a gp130-ESR1 (FIG. 12A) or a gp130-MDM4 (FIG. 12B) fusion construct, together with the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with leptin and compound (or DMSO as negative control) at 24 hours after transfection. Luciferase activity was measured 24 hours after compound treatment using the Luciferase Assay System kit (PROMEGA, Madison, Wis.) with an Ensight plate reader (PERKIN ELMER LIFE SCIENCES, Waltham, Mass.). The graphs shown in FIGS. 12A-B (left panel) depict the frequency distributions of the average raw luciferase signal for both the compound-treated samples and the DMSO-treated controls. Both distributions largely overlap, but a number of compounds exhibit a luciferase signal above background. These outliers are depicted by the line marks on the frequency curve. For each of the two exemplified screens, one hit was confirmed in dose-response analysis (right panel). These dose-response curves were generated using the same assay setup and protocol used for the primary screen (except for the use of an alternative DHFR receptor anchor fusion construct, pCLL-DHFR, which contains the mutant leptin receptor intracellular domain instead of the Gly-Gly-Ser hinge described in Example 2), but now testing a 9-point dose-range of the indicated concentrations. Here, data points depict fold induction of the average luciferase activity of triplicate samples from leptin+test compound treated cells versus leptin only treated cells. Error bars represent standard deviations and curves were fit using 4-parameter nonlinear regression in GRAPHPAD PRISM software. In the case of the ESR1 target screen, the confirmed hit corresponds to TMP-TAM, where TAM is a known ESR1 ligand and as such these data validate the MAPPIT-derivative screening approach. In summary, these examples show that the MAPPIT-derivative assay presented here can be applied to screen hybrid ligand collections to identify known and novel ligand-target interactions.

### Example 12: Cell Microarray-Based ORF cDNA Library Screening with a MAPPIT-Derivative Assay to Identify Novel Hybrid Ligand Targets

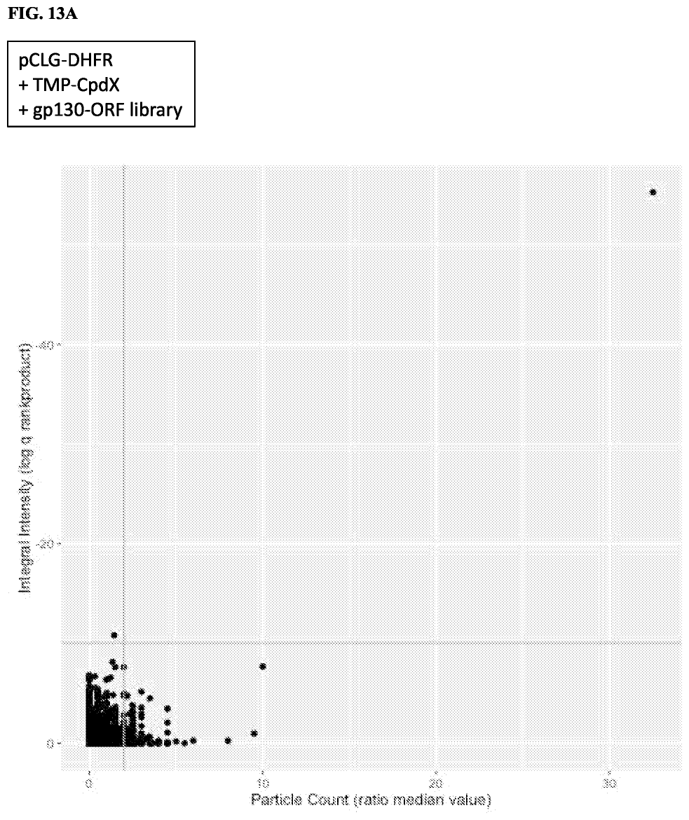

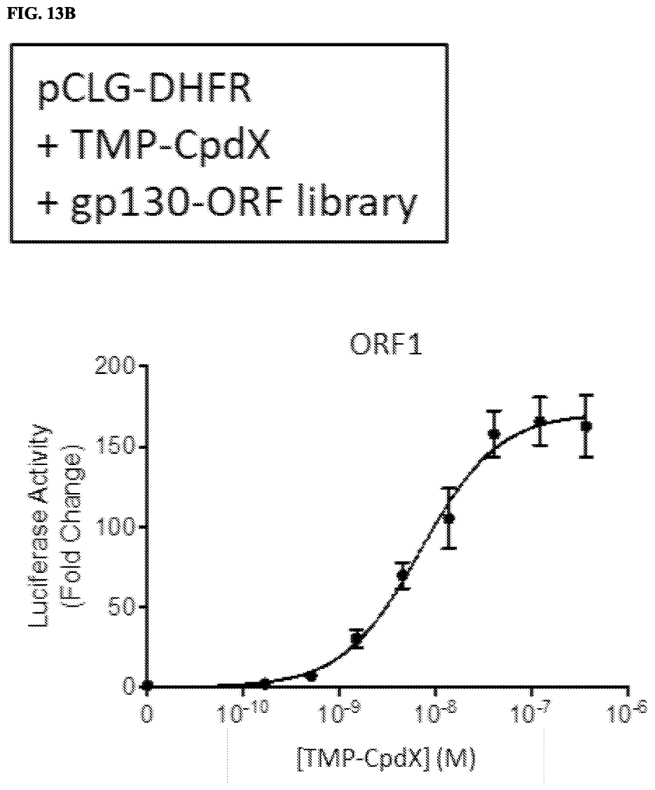

In this Example, in order to identify novel target proteins of hybrid ligand bait molecules, a MAPPIT cell microarray screen was performed similar to the one described in Example 10, using the procedure described in Lievens, et al. “Proteome-scale binary interactomics in human cells.” Molecular & Cellular Proteomics 15.12 (2016): 3624-3639. Here, this screening approach was applied to identify targets of an undisclosed compound with a strong antitumor phenotype for which no target was known. To this end, a hybrid ligand fusion compound was synthesized linking TMP with this compound through a PEG tether. HEK293T cells were transfected with the same (E. coli) DHFR receptor anchor fusion plasmid (pCLG-DHFR) that was used in the previous Example 11. These transfected cells were then added to microarray screening plates containing a prey gp130 fusion expression plasmid collection covering over 15,000 ORFs. Each spot in the microarray contained a different gp130-ORF fusion expression plasmid, as well as a STAT3-responsive fluorescence protein-encoding reporter plasmid. DHFR anchor fusion transfected cells landing and attaching on these spots therefore become transfected as well with the gp130-ORF prey plasmid and the reporter plasmid. Twenty-four hours after transfection cells were differentially stimulated with leptin with and without the TMP-compound hybrid ligand (5 μM final concentration), and reporter signal (GFP-like fluorescence reporter) was read out 48 hours later. Fluorescence intensity data was analyzed as reported previously, yielding a volcano plot where q-values calculated based on the integrated fluorescence intensity of each microarray cell cluster (Y-axis) are displayed against the ratio of the median value of the fluorescent particle count of the corresponding cell clusters (X-axis), as shown in FIGS. 13A-B. One ORF cDNA exhibited a strong signal (indicated by an arrow on the dot plot in FIGS. 13A-B) and was selected for dose-response confirmation. The DHFR receptor fusion plasmid (pCLG-DHFR) was co-transfected in HEK293T cells together with the corresponding gp130-ORF plasmid and the luciferase reporter plasmid, 24 h after transfection the cells were treated with leptin without and with the indicated concentration of hybrid ligand, and another 24 h later luciferase activity was determined. The dose-response curves represent the fold induction of the average luciferase activity of triplicate samples from leptin+test compound treated cells versus leptin only treated cells. Error bars represent standard deviations and curves were fit using 4-parameter nonlinear regression in GRAPHPAD PRISM software. This example shows that the MAPPIT-derivative assay presented here can be applied to screen ORF cDNA collections to identify novel ligand protein targets.

### Example 13: Detection of Rapamycin-Induced Recruitment of MTOR to FKBP Proteins

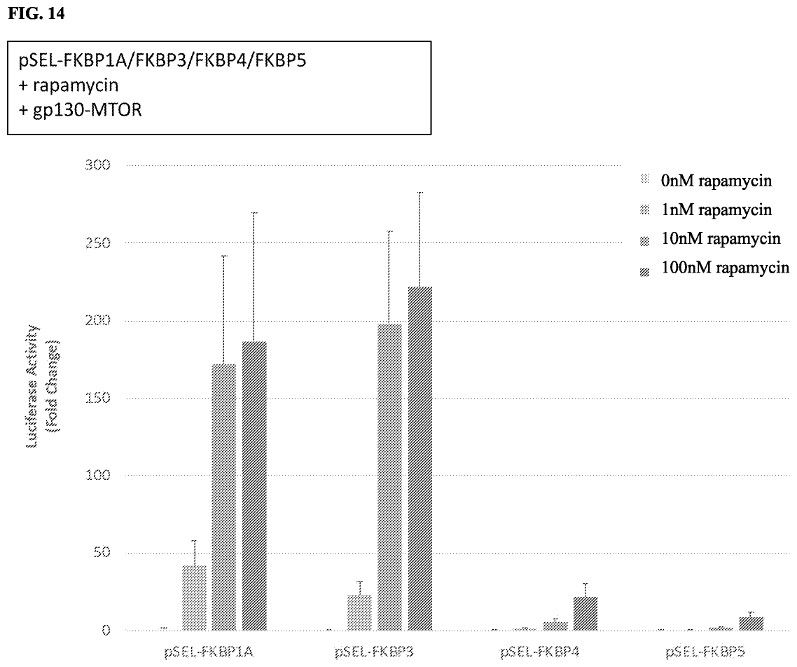

In this Example, MAPPIT-derivative assays were developed to monitor rapamycin-induced binding between MTOR and FKBP protein family members, specifically FKBP1A (FKBP12), FKBP3, FKBP4 and FKBP5. As indicated in FIG. 14, the FKBP cDNAs were cloned as MAPPIT receptor fusions containing the EPO receptor extracellular domain (pSEL-FKBPx) and MTOR (FRB domain) was cloned as a gp130 fusion. HEK293T cells were co-transfected with any of the FKBP receptor fusion constructs together with the gp130-MTOR fusion plasmid and the STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Cells were treated with EPO without or with the indicated dose of rapamycin at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depict fold induction of the average luciferase activity of triplicate samples from EPO+test compound treated cells versus EPO or leptin only treated cells. Error bars represent standard deviations. As shown, a rapamycin-induced reporter signal could be obtained for each of the FKBP-MTOR interactions, as reported previously in the literature.

What is claimed:

1. A method for detecting a molecular interaction, comprising:
(a) providing a cell comprising a ligand-dependent chimeric receptor comprising:
(i) an extracellular portion of a ligand-binding domain derived from a first receptor and
(ii) transmembrane and cytoplasmic domains of a second receptor and a intracellular bait protein fused thereto, wherein the transmembrane and/or cytoplasmic domains of the second receptor comprise mutations that reduce or eliminate STAT (Signal Transducer and Activator of Transcription) recruitment;
(b) expressing a prey protein that is fused to a receptor fragment in the cell, the receptor fragment comprising functional STAT recruitment sites; and
(c) detecting a signal that is indicative of a molecular interaction,
wherein, the bait protein is an E3 ligase substrate binding subunit.

2. A method for detecting a molecular interaction, comprising:
(a) providing a cell comprising a ligand-dependent chimeric receptor comprising:
(i) an extracellular portion of a ligand-binding domain derived from a first receptor and
(ii) transmembrane and cytoplasmic domains of a second receptor and a intracellular bait scaffold protein fused thereto, wherein the transmembrane and/or cytoplasmic domains of the second receptor comprise mutations that reduce or eliminate STAT (Signal Transducer and Activator of Transcription) recruitment;
(b) expressing a prey protein that is fused to a receptor fragment in the cell, the receptor fragment comprising functional STAT recruitment sites; and
(c) detecting a signal that is indicative of a molecular interaction,
wherein, the bait scaffold protein fused to the transmembrane and/cytoplasmic domain of the second receptor is associated with a bait protein that is an E3 ligase substrate binding subunit.

3. The method of claim 1 or 2, wherein the interaction between the prey protein and bait protein causes recruitment of the receptor fragment fused to the bait protein to the transmembrane chimeric receptor protein, which restores ligand-dependent transmembrane chimeric receptor signaling and activation of STAT molecules.

4. The method of claim 3, wherein the cell comprises a STAT-responsive reporter gene.

5. The method of claim 4, wherein the activated STAT molecules migrate to the nucleus and induce transcription of the STAT-responsive reporter gene, the reporter gene signal permitting detection of a molecular interaction.

6. The method of any one of claims 1-5, wherein the E3 ligase substrate binding subunit is selected from cereblon (CRBN) and Von Hippel Lindau (VHL).

7. The method of any one of claims 2-6, wherein the scaffold protein is selected from damaged DNA binding protein 1 (DDB1), Cullin-4A (CUL4A), regulator of cullins 1 (ROC1), SKIP1, SKP1 interacting partner (SKIP2), Beta-transducin repeats-containing protein (β-TrCP), Double minute 4 protein (MDM4), X-Linked Inhibitor of Apoptosis (XIAP), DDB1 And CUL4 Associated Factor 15 (DCAF15), and WD Repeat Domain 12 (WDR12).

8. The method of any one of the above claims, wherein the method further comprises introducing a small molecule which binds to the prey protein and/or bait protein.

9. The method of claim 8, wherein the molecular interaction is a protein/protein interaction which is mediated by the binding of the small molecule with the prey protein or bait protein.

10. The method of any one of the above claims, wherein the molecular interaction is two or more protein/protein interactions which are mediated by the binding of the small molecule with the prey protein or bait protein.

11. The method of any one of claims 8-10, wherein the protein/protein interaction which is mediated by the binding of the small molecule with the prey protein or bait protein is a direct binding between the prey protein or bait protein and the small molecule at a protein/protein interface.

12. The method of any one of claims 8-10, wherein the protein/protein interaction which is mediated by the binding of the small molecule with the prey protein or bait protein is mediated by an allosteric modification of the protein surface of the bait protein.

13. The method of claim 12, wherein the small molecule induces exposure of a hydrophobic surface of the bait protein that allows for interaction with the prey protein.

14. The method of any one of claims 8-13, wherein the small molecule is a molecular glue.

15. The method of claim 1 or 2, wherein the molecular interaction is a complex formation.

16. The method of claim 1 or 2, wherein the molecular interaction is a small molecule/protein interaction.

17. The method of any one of claims 1-16, wherein the first receptor and second receptor are the same.

18. The method of any one of claims 1-16, wherein the first receptor and second receptor are different.

19. The method of any one of claims 1-18, wherein the first receptor and/or second receptor is a multimerizing receptor.

20. The method of any one of claims 1-19, wherein the ligand-binding domain is derived from a cytokine receptor.

21. The method of any one of claims 1-20, wherein the ligand-binding domain is derived from a Type 1 cytokine receptor (CR).

22. The method of any one of claims 1-20, wherein the ligand-binding domain is derived from erythropoietin receptor (EpoR) or leptin receptor (LR).

23. The method of claim 22, wherein the transmembrane and cytoplasmic domains are derived from the murine leptin receptor (LR).

24. The method of any one of claims 1-23, wherein the bait is heterologous to the first receptor and/or second receptor fragment.

25. The method of any one of claims 1-24, wherein the cytoplasmic domain comprises a JAK binding site and/or the receptor fragment comprises gp130.

26. The method of any one of claims 1-25, wherein the STAT is selected from STAT1 or STAT3.

27. The method of any one of claims 1-26, wherein the mutations that reduce or eliminate STAT recruitment are to one or more tyrosine phosphorylation sites.

28. The method of any one of claims 1-27, wherein the transmembrane and cytoplasmic domains are derived from the murine leptin receptor (LR) and the mutations are at one or more of positions Y985, Y1077, and Y1138.

29. The method of any one of claims 1-28, wherein the transmembrane and cytoplasmic domains are derived from the murine leptin receptor (LR) and the mutations are Y985F, Y1077F, and Y1138F.

30. The method of any one of claims 1-29, wherein the transmembrane and cytoplasmic domains have functionally equivalent mutations to Y985F, Y1077F, and Y1138F of the murine leptin receptor (LR).

31. The method of any one of claims 1-30, wherein the prey protein comprises a nuclear export sequence (NES).

32. The method of claim 31, wherein the NES has 1˜4 hydrophobic residues.

33. The method of claim 32, wherein the hydrophobic residues are leucines.

34. The method of any one of claims 32-33, wherein the NES has the sequence LxxxLxxLxL, where L is a hydrophobic residue and x is any other amino acid.

35. The method of any one of claims 32-34, wherein the NES has the sequence LxxxLxxLxL, where L is a leucine and x is any other amino acid.

36. The method of any of the above claims, where the bait is contacted with a compound before interaction with the prey protein.

37. The method of claim 36, wherein the compound comprises a glutarimide ring and a phthalimide ring.

38. The method of claim 37, wherein the compound is selected from thalidomide, lenalidomide, pomalidomide, CC-220, CC-122, CC-885, or a derivative or analog thereof or a compound that binds to the same CRBN bait binding site as the thalidomide, lenalidomide, pomalidomide, CC-220, CC-122, CC-885, or a derivative or analog thereof and in a competitive fashion.

39. The method of any of the above claims, wherein the method identifies:
a novel protein/protein interaction which is mediated by the binding of the small molecule with the prey protein or bait protein or
a small molecule compound that induces, mediates or stabilizes a protein-protein interaction that comprises the prey protein and bait protein, the small molecule compound optionally being a molecular glue or hybrid ligand.

40. A method for detecting a molecular interaction, comprising:
(a) providing a cell comprising a ligand-dependent chimeric receptor comprising:
(i) an extracellular portion of a ligand-binding domain derived from a first receptor and
(ii) transmembrane and cytoplasmic domains of a second receptor and a intracellular bait protein fused thereto, wherein the transmembrane and/or cytoplasmic domains of the second receptor comprise mutations that reduce or eliminate STAT (Signal Transducer and Activator of Transcription) recruitment;
(b) expressing a prey protein that is fused to a receptor fragment in the cell, the receptor fragment comprising functional STAT recruitment sites; and
(c) detecting a signal that is indicative of a molecular interaction,
wherein, the bait protein is an FK506 binding protein (FKBP).

41. The method of claim 40, wherein the interaction between the prey protein and bait protein causes recruitment of the receptor fragment fused to the bait protein to the transmembrane chimeric receptor protein, which restores ligand-dependent transmembrane chimeric receptor signaling and activation of STAT molecules.

42. The method of claim 41, wherein the cell comprises a STAT-responsive reporter gene.

43. The method of claim 42, wherein the activated STAT molecules migrate to the nucleus and induce transcription of the STAT-responsive reporter gene, the reporter gene signal permitting detection of a molecular interaction.

44. The method of any one of claims 40-43, wherein the FK506 binding protein (FKBP) is selected from FKBP12, FKBP38 and FKBP52.

45. The method of any one of claims 40-44, wherein the method further comprises introducing a small molecule which binds to the prey protein and/or bait protein.

46. The method of claim 45, wherein the molecular interaction is a protein/protein interaction which is mediated by the binding of the small molecule with the prey protein or bait protein.

47. The method of any one of claims 40-46, wherein the molecular interaction is two or more protein/protein interactions which are mediated by the binding of the small molecule with the prey protein or bait protein.

48. The method of any one of claims 45-47, wherein the protein/protein interaction which is mediated by the binding of the small molecule with the prey protein or bait protein is a direct binding between the prey protein or bait protein and the small molecule at a protein/protein interface.

49. The method of any one of claims 45-47, wherein the protein/protein interaction which is mediated by the binding of the small molecule with the prey protein or bait protein is mediated by an allosteric modification of the protein surface of the bait protein.

50. The method of claim 49, wherein the small molecule induces exposure of a hydrophobic surface of the bait protein that allows for interaction with the prey protein.

51. The method of any one of claims 45-50, wherein the small molecule is a molecular glue.

52. The method of claim 40 or 41, wherein the molecular interaction is a complex formation.

53. The method of claim 40 or 41, wherein the molecular interaction is a small molecule/protein interaction.

54. The method of any one of claims 40-53, wherein the first receptor and second receptor are the same.

55. The method of any one of claims 40-54, wherein the first receptor and second receptor are different.

56. The method of any one of claims 40-55, wherein the first receptor and/or second receptor is a multimerizing receptor.

57. The method of any one of claims 40-56, wherein the ligand-binding domain is derived from a cytokine receptor.

58. The method of any one of claims 40-57, wherein the ligand-binding domain is derived from a Type 1 cytokine receptor (CR).

59. The method of any one of claims 40-57, wherein the ligand-binding domain is derived from erythropoietin receptor (EpoR) or leptin receptor (LR).

60. The method of claim 59, wherein the transmembrane and cytoplasmic domains are derived from the murine leptin receptor (LR).

61. The method of any one of claims 40-60, wherein the bait is heterologous to the first receptor and/or second receptor fragment.

62. The method of any one of claims 40-61, wherein the cytoplasmic domain comprises a JAK binding site and/or the receptor fragment comprises gp130.

63. The method of any one of claims 40-62, wherein the STAT is selected from STAT1 or STAT3.

64. The method of any one of claims 40-63, wherein the mutations that reduce or eliminate STAT recruitment are to one or more tyrosine phosphorylation sites.

65. The method of any one of claims 40-64, wherein the transmembrane and cytoplasmic domains are derived from the murine leptin receptor (LR) and the mutations are at one or more of positions Y985, Y1077, and Y1138.

66. The method of any one of claims 40-65, wherein the transmembrane and cytoplasmic domains are derived from the murine leptin receptor (LR) and the mutations are Y985F, Y1077F, and Y1138F.

67. The method of any one of claims 40-66, wherein the transmembrane and cytoplasmic domains have functionally equivalent mutations to Y985F, Y1077F, and Y1138F of the murine leptin receptor (LR).

68. The method of any one of claims 40-67, wherein the prey protein comprises a nuclear export sequence (NES).

69. The method of claim 68, wherein the NES has 1˜4 hydrophobic residues.

70. The method of claim 69, wherein the hydrophobic residues are leucines.

71. The method of any one of claims 69-70, wherein the NES has the sequence LxxxLxxLxL, where L is a hydrophobic residue and x is any other amino acid.

72. The method of any one of claims 69-71, wherein the NES has the sequence LxxxLxxLxL, where L is a leucine and x is any other amino acid.

73. The method of any of claims 40-72, where the bait is contacted with a compound before interaction with the prey protein.

74. The method of claim 73, wherein the compound is selected from FK506 (tacrolimus), rapamycin (sirolimus), and cyclosporin A (CsA) or a derivative or analog thereof or a compound that binds to the same FKBP bait binding site as the FK506 (tacrolimus), rapamycin (sirolimus), and cyclosporin A (CsA) or a derivative or analog thereof and in a competitive fashion.

75. The method of any of claims 40-74, wherein the method identifies a novel protein/protein interaction which is mediated by the binding of the small molecule with the prey protein or bait protein.

76. A method for detecting a molecular interaction, comprising:
(a) providing a cell comprising a ligand-dependent chimeric receptor comprising:
(i) an extracellular portion of a ligand-binding domain derived from a first receptor and
(ii) transmembrane and cytoplasmic domains of a second receptor and a intracellular bait protein fused thereto, wherein the transmembrane and/or cytoplasmic domains of the second receptor comprise mutations that reduce or eliminate STAT (Signal Transducer and Activator of Transcription) recruitment,
wherein, the bait protein is cereblon (CRBN) or FK506 binding protein (FKBP);
(b) expressing a prey protein that is fused to a receptor fragment in the cell, the receptor fragment comprising functional STAT recruitment sites;
(c) detecting a signal that is indicative of a molecular interaction; and
(d) introducing a small molecule which binds to the prey protein and/or bait protein,
wherein the molecular interaction is a protein/protein interaction which is mediated by the binding of the small molecule with the prey protein or bait protein.

77. The method of claim 76, wherein the protein/protein interaction which is mediated by the binding of the small molecule with the prey protein or bait protein is mediated by an allosteric modification of the protein surface of the bait protein.

78. The method of claim 77, wherein the small molecule induces exposure of a hydrophobic surface of the bait protein that allows for interaction with the prey protein.

79. The method of claim 78, wherein the small molecule is a molecular glue compound or hybrid ligand.

80. The method of claim 78, wherein the method identifies:
a novel protein/protein interaction which is mediated by the binding of the small molecule with the prey protein or bait protein or
a small molecule compound that induces, mediates or stabilizes a protein-protein interaction that comprises the prey protein and bait protein, the small molecule compound optionally being a molecular glue or hybrid ligand.


SEQUENCE LISTING (1 sequence)

SEQ ID NO: 1 - protein, 1162 aa, Artificial Sequence
  Synthetic polymer.
MMCQKFYVVLLHWEFLYVIAALNLAYPISPWKFKLFCGPPNTTDDSFLSPAGAPNNASALKGASEAIVEAKFNSSGIYVPELSKTVFHCCFGNEQGQNCSALTDNTEGKTLASVVKASVFRQLGVNWDIECWMKGDLTLFICHMEPLPKNPFKNYDSKVHLLYDLPEVIDDSPLPPLKDSFQTVQCNCSLRGCECHVPVPRAKLNYALLMYLEITSAGVSFQSPLMSLQPMLVVKPDPPLGLHMEVTDDGNLKISWDSQTMAPFPLQYQVKYLENSTIVREAAEIVSATSLLVDSVLPGSSYEVQVRSKRLDGSGVWSDWSSPQVFTTQDVVYFPPKILTSVGSNASFHCIYKNENQIISSKQIVWWRNLAEKIPEIQYSIVSDRVSKVTFSNLKATRPRGKFTYDAVYCCNEQACHHRYAELYVIDVNINISCETDGYLTKMTCRWSPSTIQSLVGSTVQLRYHRRSLYCPDSPSIHPTSEPKNCVLQRDGFYECVFQPIFLLSGYTMWIRINHSLGSLDSPPTCVLPDSVVKPLPPSNVKAEITVNTGLLKVSWEKPVFPENNLQFQIRYGLSGKEIQWKTHEVFDAKSKSASLLVSDLCAVYVVQVRCRRLDGLGYWSNWSSPAYTLVMDVKVPMRGPEFWRKMDGDVTKKERNVTLLWKPLTKNDSLCSVRRYVVKHRTAHNGTWSEDVGNRTNLTFLWTEPAHTVTVLAVNSLGASLVNFNLTFSWPMSKVSAVESLSAYPLSSSCVILSWTLSPDDYSLLYLVIEWKILNEDDGMKWLRIPSNVKKFYIHDNFIPIEKYQFSLYPVFMEGVGKPKIINGFTKDAIDKQQNDAGLYVIVPIIISSCVLLLGTLLISHQRMKKLFWDDVPNPKNCSWAQGLNFQKPETFEHLFTKHAESVIFGPLLLEPEPISEEISVDTAWKNKDEMVPAAMVSLLLTTPDPESSSICISDQCNSANFSGSQSTQVTCEDECQRQPSVKYATLVSNDKLVETDEEQGFIHSPVSNCISSNHSPLRQSFSSSSWETEAQTFFLLSDQQPTMISPQLSFSGLDELLELEGSFPEENHREKSVCYLGVTSVNRRESGVLLTGEAGILCTFPAQCLFSDIRILQERCSHFVENNLSLGTSGENFVPYMPQFQTCSTHSHKIMENKMCDLTV